(19) United States
(12) Patent Application Publication
(10) Pub. No.: US 2009/0005385 A1
(43) Pub. Date: Jan. 1, 2009

(54) Treatment or Prevention of Cancer or Cardiovascular Disease with Methenyltetrahydrofolate Synthetases

(75) Inventor: Patrick J. Stover, Ithaca, NY (US)

Correspondence Address: NIXON PEABODY LLP - PATENT GROUP, 1100 CLINTON SQUARE, ROCHESTER, NY, 14604, US

(73) Assignee: CORNELL RESEARCH FOUNDATION, INC., Ithaca, NY (US)

(21) Appl. No.: 11/571,213

(22) PCT Filed: Jun. 13, 2005
(86) PCT No.: PCT/US05/20847

§ 371 Date: Feb. 20, 2007

Related U.S. Application Data
(60) Provisional application No. 60/585,293, filed on Jul. 2, 2004.

(51) Int. Cl. A61K 31/495; C12Q 1/02; A61P 31/00

(52) U.S. Cl. 514/249; 435/29

(57) ABSTRACT

The present invention relates to a method of screening test substances for chemotherapeutic activity or for efficacy in treating cardiovascular disease by providing one or more cells transformed with a nucleic acid molecule encoding methenyltetrahydrofolate synthetase, contacting the cells with test substance(s), and identifying those test substances which modulate methenyltetrahydrofolate synthetase expression as candidates for such therapeutic use. Another aspect of the present invention relates to a method of measuring folate status in a sample by measuring methenyltetrahydrofolate synthetase expression or activity and/or catecholamine activity in the sample, all of which are correlated to folate status in the sample. The present invention can also be used to treat or prevent cancer or cardiovascular disease in a subject by administering to the subject a substance which modulates methenyltetrahydrofolate synthetase expression or activity and/or catecholamine activity.

This application claims the benefit of U.S. Provisional Patent Application Ser. No. 60/585,293, filed Jul. 2, 2004.

The subject matter of this application was made with support from the United States Government under National Institutes of Health Grant No. HD35687-01. The U.S. Government may have certain rights.

### FIELD OF THE INVENTION

The present invention relates to a method of screening test compounds for chemotherapeutic activity or efficacy in treating cardiovascular disease, a method of measuring folate status, a method of treating or preventing cancer, and a method of treating or preventing cardiovascular disease.

### BACKGROUND OF THE INVENTION

### Nutrition and Cancer.

Nutrition is one of the most important and easily manipulated environmental factors associated with both cancer risk and prevention (Goldgar et al., “Systematic Population-based Assessment of Cancer Risk in First-degree Relatives of Cancer Probands,” J. Natl. Inst 86:1600-1608 (1994)). Studies demonstrate that over one third of all cancer deaths are attributable to diet, compared to 5-10% of all cancers that result almost exclusively from heredity including “highly penetrate single-gene mutations” and dominant genetic factors. Risk for the later is most associated with early onset cancer, whereas cancers of the stomach, lung, pancreas, ovary, bladder and leukemia have limited genetic causalty (Lichtenstein et al., “Enviromental and Heritable Factors in the Causation of Cancer: Analyses of Cohorts of Twins from Sweden, Denmark and Finland,” N. Engl. J. Med. 343:78-85 (2000)). The relative contribution of genetics and environment in variety of sporadic cancers has been determined by examining the concordance of cancer between monozygotic twins, who share common genetic backgrounds, and dizygotic twins, who share on average 50% of segregating genes. While the nature of the environmental effects is population specific, heritable factors range from 27 to 42% for colorectal, breast and prostate cancer (Doll et al. “The Causes of Cancer: Quantitative Estimates of Avoidable Risks of Cancer in the United States Today,” J. Natl. Cancer Inst. 66:1191-1308 (1981)). These data indicate that environment, including nutrition, is a major contributor to sporadic cancer. Similar conclusions may be inferred from migration studies of populations who maintain their genetic backgrounds and from longitudinal studies of resident populations who maintain genetic backgrounds but whose diets undergo dramatic changes (Kaur, “Migration Patterns and Breast Carcinoma,” Cancer 88:1203-1206 (2000)). The key for advancing the mechanisms associated with sporadic cancer must not only consider the effects of nutrients and genetics on cancer, but the modifying effects that they exert on each other. General mechanisms of carcinogenesis. As recently reviewed, “age is the most potent of all carcinogens” (DePinho, “The Age of Cancer,” Nature 408:248-254 (2000)). Cancer risk rises exponentially after the age of 40, driven primarily by an increase in the incidence of epithelial carcinomas. Several mechanisms have been proposed to account for age-related cancer susceptibility, including increased mutational load and epigenetic gene silencing. Mutational load refers to the cumulative acquisition of somatic mutations in DNA that arise from DNA damaging agents, background rates of error associated with DNA replication and repair, and declining DNA repair efficiency. As mutational load increases, particularly in key loci that encode genes critical for cell-cycle control, cancer is initiated and progresses through the process of clonal selection. Epigenetic mechanisms, namely DNA methylation, alterations in chromatin structure, and the effects of methylation on chromatin structure, have also been proposed to contribute to both the initiation and progression of certain cancers. Recent microarray analyses have indicated that about 10% of murine genes are regulated by DNA methylation (Jackson-Grusby et al., “Loss of Genomic Methylation Causes p53-dependent Apoptosis and Epigenetic Silencing,” Nature Genetics 27:31-39 (2001)). DNA methylation increases as a function of age in a tissue-specific manner, and methylation density within the 5′ promoter region correlates inversely with levels of gene expression for most methylation sensitive genes (Zingg et al., “Genetic and Epigenetic Aspects of DNA Methylation on Genome Expression, Evolution, Mutation and Carcinogenesis,” Carcinogenesis 18:869-882 (1997)). Tumor-suppressor genes seem to be particularly sensitive to methylation silencing, and their silencing occurs concurrently with tumor progression. The relationship between DNA methylation and cancer susceptibility is supported in murine models in which impaired activity of the cytosine methyltransferase (Dnmt1) reduced genome wide methylation density and reduced the number of age-accumulated adenomas in Min (APC deficient) mice (Laird et al., “Suppression of Intestinal Neoplasia by DNA Hypomethylation,” Cell 81:197-205 (1995)).

### Folate and Carcinogenesis.

The importance of folate-mediated one-carbon metabolism in fundamental metabolic and cellular processes, including DNA synthesis and methylation has been recognized since the 1940s. Almost immediately following the discovery of folate as a metabolic cofactor, folate analogs or antifolates were developed which proved to be effective antimicrobial and antineoplasic agents. Folate antagonists were the first antimetabolite anticancer agents and one of the first modern anticancer drugs (Zhao et al., Oncogene 22:7431-57 (2003)). To date, the folate-dependent enzymes dihydrofolate reductase (DHFR), thymidylate synthase (TS), and glycinamide ribonucleotide formyltransferase (GARFT) have been targets for inhibitor design including 5-fluorouracil, methotrexate, 5,8-dideazatetrahydrofolate and Tomudex (raltitrexed). All of these antifolates target enzymes required for DNA synthesis in the cytoplasm, and their effectiveness varies among different tumor types, and depend on their cellular metabolism and accumulation.

More recent studies have established a role for dietary folate in cancer susceptibility and prevention (Ames, B. N., Proc Natl Acad Sci USA 96:12216-28 (1999); Molloy et al., Public Health Nutr 4:601-9 (2001); Blount et al., Proc Natl Acad Sci USA 94:3290-5 (1997)). Results from epidemiological, genetic, biochemical, and animal studies are revealing that impairments in folate metabolism contribute to the initiation and progression of epithelial cancers, especially colon cancer (Ueland et al., Am J Clin Nutr 72:324-32 (2000)). Impairments of folate metabolism can result not only from folate deficiency, but also from pharmaceutical therapies, genetic predisposition including single nucleotide polymorphisms, and certain physiological states.

### Proposed Mechanisms for Folate in Epithelial Cancers.

Two general mechanisms have been proposed to account for the many associations between altered folate metabolism and cancer (Kim, “Folate and Carcinogenesis: Evidence, Mechamisms, and Implications,” J. Nutr. Biochem. 10:66-88 (1999)).

### Alteration of DNA Methylation.

Approximately 4% of cytosine bases within the mammalian genome are modified by methylation, and both genome-wide and allele specific DNA methylation is influenced by folate metabolism in some but not all studies. Methylated cytosine residues are generally located in CpG islands, which are dinucleotide repeat sequences commonly found in the 5′ promoter regions of genes. Up to 90% of cytosine bases are methylated within CpG islands. Genome-wide DNA hypomethylation occurs in nearly all cancers and precedes mutational and chromosomal abnormalities that occur as cancer progresses, whereas allelic specific hypermethylation and gene silencing occurs concurrently. DNA hypomethylation, which can be induced by folate deficiency, has two primary effects on the mammalian genome. First, it increases the expression of genes normally silenced by methylation, including oncogenes genes. Second, hypomethylation relaxes chromatin structure (Antequera et al., “High Levels of De Novo Methylation and Altered Chromatin Structure at CpG Islands in Cell Lines,” Cell 62:503-514 (1990)) and thereby enhances the accessibility of DNA damaging agents resulting in increased genomic mutation, particularly in “hot spots” associated with cancers. In support of this mechanism, rodents fed diets deficient in folate and other sources of methyl groups were shown to be more susceptible to chemically induced hepatocarcinoma (Newbern e et al., “Labile Methyl Groups and the Promotion of Cancer,” Ann Rev. Nutr. 6:407-432 (1986)). Given that mutation is by definition an irreversible process, mechanisms that implicate folate in genome mutation and stability are more consistent with our accumulated knowledge of cellular transformation.

### Impaired DNA Synthesis and Repair.

Folate deficiency in cell cultures results in imbalances in the dNTP pools, increased uracil concentration in DNA, and chromosomal breaks (Branda et al., “Folate Deficiency Increases Genetic Damage Caused by Alkylating Agents and G-Irradiation in Chinese Hamster Ovary Cells,” Can. Res. 53:5401-5408 (1993)). All of these abnormalities can be reversed by folate repletion of the culture medium. Tumors also display these biochemical characteristics (Kim, “Folate and Carcinogenesis: Evidence, Mechamisms, and Implications,” J. Nutr. Biochem. 10:66-88 (1999)). In humans and rodents, folate status correlates inversely with uracil content in DNA, presumably resulting from impaired dTMP synthesis and subsequent elevations in dUTP pools. Elevation in dUTP pools likely results in its misincorporation into DNA during replication (Blount et al., “Folate Deficiency Causes Uracil Misincorporation into Human DNA and Chromosome Breakage: Implications for Cancer and Neuronal Damage,” Proc. Natl. Acad. Sci. 94:3290-3295 (1997)). Once uracil is incorporated into DNA, excision repair of uracil from DNA has the potential to result in strand breaks if the residues are in close proximity and are located on opposite strands of the helix. In vitro studies have demonstrated a linearization of plasmid DNA occurs when uracil residues are placed within circular plasmid DNA following addition of the repair enzyme, uracil DNA glycosylase (Salganik et al., “Molecular Mechanisms of the Formation of DNA Double Strand Breaks and Induction of Genomic Rearrangements,” Mutation Res. 266:163-170 (1992)). Imbalances in the dNTP pool may increase the error associated with DNA polymerase activity, as well as excision repair enzymes.

In conclusion, the effects of folate deficiency on both DNA mutation rates and alterations in genome methylation may function in concert during cellular transformation and cannot be considered in isolation during experimentation.

### Cardiovascular Disease.

Folate deficiency leads to elevated concentrations of homocysteine in the blood and urine, and is associated with an increased incidence of arterial and venous thromboembolic events (Refsum et al., Clin Chem. 50(1):3-32 (2004)). Associations between homocysteine and CVD has been found in numerous epidemiologic studies, both venous and arterial occlusive disease. The effect of tHcy and CVD risk is dose dependent and independent of other risk factors. Homocysteine impairs vascular function and blood coagulation, indicating mechanistic support for the homocysteine-cardiovascular relationship. However, little is known about the molecular mechanisms whereby folate deficiency and/or elevated homocysteine initiate or accelerate cardiovascular disease.

The present invention is directed to achieving the art's need for discoverying new agents with chemotherapeutic activity or efficacy in treating cardiovascular disease.

### SUMMARY OF THE INVENTION

One embodiment of the present application relates to a method of screening test substances for chemotherapeutic activity or efficacy in treating cardiovascular disease. This method involves providing one or more cells transformed with a nucleic acid molecule encoding methenyltetrahydrofolate synthetase and then contacting the cells with one or more test substances. Test substances which modulate methenyltetrahydrofolate synthetase expression by the cells are identified as candidate substances for chemotherapeutic activity or efficacy in treating cardiovascular disease.

Another embodiment of the present invention relates to a method of measuring folate status in a sample. This method involves providing a sample and measuring methenyltetrahydrofolate synthetase expression or activity and/or catecholamine activity in the sample. The measured level of methenyltetrahydrofolate synthetase expression or activity and/or catecholamine activity in the sample is correlated to folate status in the sample.

A further embodiment of the present invention relates to a method of treating or preventing cancer in a subject. This method involves administering to the subject a substance which modulates methenyltetrahydrofolate synthetase expression or activity and/or catecholamine activity under conditions effective to treat or prevent cancer in the subject.

Another aspect of the present invention relates to a method of treating or preventing cardiovascular disease in a subject. This method involves administering to the subject a substance which inhibits methenyltetrahydrofolate synthetase expression or activity and/or catecholamine activity under conditions effective to treat or prevent cardiovascular disease in the subject.

### BRIEF DESCRIPTION OF THE DRAWINGS

FIG. 1 shows Western Blot analyses of HCF and cSHMT in normal and tumor tissues. Biopsies of canine and feline normal and tumor tissues were obtained during surgery at Cornell University College of Veterinary Medicine. The tissue samples were sonicated, and the resulting supernatant was used to determine the amounts of HCF and cSHMT protein present by Western Analysis. The molecular mass of cSHMT is 55 kDa, and the molecular mass of HCF is 20 kDa. For all blots, only one immunoreactive band was present, and all bands corresponded to the expected molecular mass for HCF or cSHMT. The (*) indicates two independent biopsies from the same animal.

FIG. 2 shows stable isotope incorporation into one-carbon metabolism. The hydroxymethyl group of L-[2,3,3′-²H₃]serine is incorporated into one-carbon metabolism through its conversion to 5,10-methyleneTHF by the enzyme SHMT, which is expressed in the cytoplasm and the mitochondria. Mitochondrial-derived formate can enter the cytoplasm, and is incorporated into the folate-activated one-carbon pool as 10-formylTHF. MS, methionine synthase; MTHFR, methylenetetrahydrofolate reductase; cSHMT, cytoplasmic serine hydroxymethyltransferase; mSHMT, mitochondrial serine hydroxymethyltransferase; TS, thymidylate synthase; MTHFS, methenyltetrahydrofolate synthetase, SAM, S-adenosylmethionine; SAH, S-adenosylhomocysteine; Hcy, homocysteine.

FIG. 3 shows the effect of folate depletion on MTHFS and HCF protein levels in MCF-7 and SH-SY5Y cell lines. Cells were cultured to 70% confluence in defined αMEM media for a total of 7 days, then harvested by trypsinization and lysed. The levels of MTHFS and HCF protein in the cell lysates were determined by Western Analysis. The immunoreactive band corresponding to MTHFS (23 kDa) is indicated by the arrow.

FIG. 4 shows the UV spectrum of recombinant mouse MTHFS protein. Recombinant mouse MTHFS protein, expressed in E. coli BL21 Star™ cells grown in LB media, was purified using metal affinity chromatography then dialyzed overnight in 100 mM Tris pH 8.0. Recombinant mouse MTHFS (solid line) has an absorbance shoulder peak at λₘₐₓ=320 nm that is absent in purified rabbit liver MTHFS protein (dashed line).

FIG. 5 shows the MALDI-TOF spectrum of recombinant mouse MTHFS protein. The molecular mass of recombinant mouse MTHFS was determined by MALDI-TOF mass spectrometry. A sample of the recombinant mouse MTHFS protein in water was diluted 1:1 with 100% acetonitrile, added to a sinapinic acid matrix, and then spotted on the laser target. The recombinant mouse MTHFS protein is present as two predominant species, containing (m/z=25403.249) or lacking the chromophore (m/z=25221.610). Horse apomyoglobin (m/z=16951) was added to the MTHFS protein sample as an internal standard. The inset figure is an enlargement of the peaks corresponding to recombinant mouse MTHFS protein.

FIGS. 6A-B show that the UV spectrum of the chromophore removed from recombinant mouse MTHFS resembles the spectrum of oxidized NADA pH 7.0. The chromophore was removed from the recombinant mouse MTHFS protein using a 5-formylTHF-Sepharose column equilibrated with 20 mM Tris pH 7.0. Fractions of the flowthrough were collected immediately after the protein was added. FIG. 6A shows the UV spectrum of the flowthrough fraction containing the chromophore removed from recombinant mouse MTHFS protein. FIG. 6B shows the UV spectrum for oxidized NADA pH 7.0.

FIGS. 7A-C show the positive and negative mass spectra for the chromophore removed from the recombinant mouse MTHFS protein. The method used to remove the chromophore from recombinant mouse MTHFS protein generates different mass spectra. For spectra A and B, recombinant mouse MTHFS protein was purified using affinity chromatography, then applied to a 5-formylTHF-sepharose affinity column equilibrated with 20 mM 2-mercaptoethanol. Fractions of the flowthrough were collected, and concentrated using a speed vac. FIG. 7A shows the positive mass spectrum for the chromophore. FIG. 7B shows the negative mass spectrum for the chromophore. The peaks labeled with asterisks are an exact match to oxidized NADA standards. The peaks labeled with crosses differ from oxidized NADA standards by one atomic mass unit. The spectra for oxidized NADA standards are not shown. FIG. 7C shows the chromophore was removed from recombinant mouse MTHFS protein by acetonitrile extraction and analyzed using ESI-MS. The positive mass spectrum of the chromophore is shown.

FIGS. 8A-B show the ESI analysis of the chromophore removed from recombinant mouse MTHFS protein expressed in E. coli grown in M9 minimal media containing NADA. E. coli expressing recombinant mouse MTHFS protein were grown in M9 minimal medium, and the MTHFS protein was purified as described previously (Suh et al., “Purification and Properties of a Folate-Catabolizing Enzyme,” Journal of Biological Chemistry 275:35646-35655 (2000), which is hereby incorporated by reference in its entirety). The purified mouse MTHFS protein was loaded on a 5-formylTHF-sepharose column and flowthrough fractions were collected and analyzed using ESI-MS. FIG. 8A shows the spectrum of the flowthrough fractions collected from an MTHFS protein sample that was expressed in E. coli grown in M9 media. FIG. 8B shows the spectrum of the chromophore (collected as the flowthrough fractions) removed from the MTHFS protein that was expressed in E. coli grown in M9 media containing 10 mg NADA.

FIGS. 9A-B shows the reconstitution of purified rabbit liver MTHFS with oxNADA. The UV spectra of purified rabbit liver MTHFS and yeast aldehyde dehydrogenase following incubation with NADA and oxidized NADA are shown. Stoichiometric amounts of either purified rabbit liver MTHFS or yeast aldehyde dehydrogenase were incubated with NADA (dashed line), oxidized NADA pH 7.0 (solid line), and a potassium phosphate buffer pH 7.2 (dotted line), then dialyzed overnight. The UV spectra of the proteins were taken after overnight dialysis. The spectra for purified rabbit liver MTHFS is shown in FIG. 9A, and the spectra for aldehyde dehydrogenase is shown in FIG. 9B.

FIGS. 10A-B show the HPLC analysis of MTHFS-mediated folate catabolism of (6S)-[³H]-5-methylTHF. Recombinant mouse MTHFS protein (50 μM) was incubated with 50 μM CuCl₂ and oxidized NADA (50 μM) for 15 min, then dialyzed for 4-5 h against 2 L 50 mM Tris pH 8.0 to remove unbound Cu²⁺ and oxidized NADA. Prior to HPLC analysis, samples were spiked with unlabeled pABG, 5-formylTHF, and 5-methylTHF, then flash frozen at −80° C. Fractions (1 mL) were collected by HPLC, and the tritium quantified. (6S)-[³H]-5-methylTHF is present in fractions 37-42, and (6S)-[³H]-5-methylDHF corresponds to fractions 5-8. In FIG. 10A, a sample of (6S)-[³H]-5-methylTHF was added to the dialyzed MTHFS protein and incubated for 5 min at room temperature, then analyzed immediately by HPLC. The peak corresponding to pABG is labeled with an arrow. In FIG. 10B, a sample of (6S)-[³H]-5-methylTHF was added to a sample of the dialysis buffer, and incubated for 5 min at room temperature. This experiment was repeated five times, yielding similar results as those shown here for each set of reactions.

FIGS. 11A-B show the effect of NADA, dopamine, and L-DOPA on folate turnover in MCF-7 cells. Cells were labeled with 25 nM [³H]5-formylTHF for 12 h (pulse). The labeled folate was chased with defined αMEM culture medium that contained unlabeled 2 μM folic acid. Total [³H]folate compounds remaining in the cells were determined at various time points. In FIG. 11A, squares are cells chased with 2 μM folic acid, triangles are cells chased with medium containing folic acid and 20 μM dopamine, and inverted triangles are cells chased with medium containing folic acid and 20 μM NADA. In FIG. 11B, squares are cells chased with 2 μM folic acid and triangles are cells chased with medium containing folic acid and 20 μM L-dopa. All data are expressed as % cpm recovered in cells relative to the total counts recovered in cell lysates and media at each time point. All values represent triplicate measures and error bars are standard error of the mean.

FIG. 12A-B show the effect of NADA, dopamine, and epinephrine on folate accumulation and cell viability in MCF-7 cells. MCF-7 cells were incubated with 25.0 nM (6S)-[³H]5-formylTHF and various concentrations of NADA, dopamine, and epinephrine for 12 h, then viable cells were quantified using trypan blue staining. FIG. 12A shows folate accumulation in MCF-7 cells treated with NADA (inverted triangles), dopamine (squares), and epinephrine (triangles). All values were normalized to cpm for untreated cells, and untreated cells were given an arbitrary value of 100%. FIG. 12B shows cell viability (squares) and intracellular [³H]folate accumulation of viable cells (triangles) for MCF-7 cells treated with NADA. Cell viability is expressed as the percent of viable cells divided by the total number of cells. Total [³H]folate compounds were quantified in cell lysates by liquid scintillation. All values represent quadruplicate measures, and error bars are standard deviations of the mean.

### DETAILED DESCRIPTION OF THE INVENTION

One embodiment of the present application relates to a method of screening test substances for chemotherapeutic activity or efficacy in treating cardiovascular disease. This method involves providing one or more cells transformed with a nucleic acid molecule encoding methenyltetrahydrofolate synthetase and then contacting the cells with one or more test substances. Test substances which modulate methenyltetrahydrofolate synthetase expression or activity by the cells are identified as candidate substances for chemotherapeutic activity or efficacy in treating cardiovascular disease.

Impairments in folate metabolism are associated with the initiation and/or progression of cancer and cardiovascular disease, and, therefore, interventions that correct the metabolic imbalances have potential for use in medical therapies. It has been shown that alterations in MTHFS activity and expression modulate folate-dependent metabolic pathways, and therefore agents, both chemical (small molecules or nucleic acid polymers (DNA)) and dietary, that affect MTHFS expression and/or activity can be used to correct metabolic impairments associated with disease. The generation of genetically-modified model systems (bacteria, yeast, human cell lines, animals) with alterations in MTHFS expression are valuable tools to: (1) assess the effects of altered MTHFS on metabolism, biomarkers of impaired metabolism and pathology and (2) to use these model systems to investigate the effects of test compounds that alter MTHFS expression and/or activity on outcomes listed in (1).

By use of this method, a test substance can be screened for chemotherapeutic activity or for efficacy in treating cardiovascular disease.

In this aspect of the present invention, a recombinant methenyltetrahydrofolate synthetase protein is inserted into the host, for example, E. coli, to generate a transgenic host harboring a nucleic acid molecule encoding methenyltetrahydrofolate synthetase. To isolate the desired protein, the E. coli host cell carrying a recombinant plasmid is propagated, homogenized, and the homogenate is centrifuged to remove bacterial debris. The supernatant is then subjected to sequential ammonium sulfate precipitation. The fraction containing the desired protein of the present invention is subjected to gel filtration in an appropriately sized dextran or polyacrylamide column to separate the proteins. If necessary, the protein fraction may be further purified by HPLC or other chromatography techniques, for example, metal affinity chromatography. Alternative methods known in the art may be used as suitable.

In this aspect of the present invention, a nucleic acid molecule encoding methenyltetrahydrofolate synthetase can be introduced into an expression system or vector of choice using conventional recombinant technology. Generally, this involves inserting the nucleic acid molecule into an expression system to which the molecule is heterologous (i.e., not normally present). However, insertion of the nucleic acid molecule into an expression system in which the nucleic acid is normally expressed can also be useful to increase expression of the protein. The heterologous nucleic acid molecule is inserted into the expression system or vector in proper sense (5′→3′) orientation and correct reading frame. The vector contains the necessary elements for the transcription and translation of the inserted protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety).

A variety of host-vector systems may be utilized to express the recombinant methenyltetrahydrofolate synthetase protein or polypeptide. Primarily, the vector system must be compatible with the host cell used. Host-vector systems include, but are not limited to, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation).

Transcription of DNA is dependent upon the presence of a promoter, which is a DNA sequence that directs the binding of RNA polymerase, and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

A nucleic acid molecule(s) encoding methenyltetrahydrofolate synthetase, a promoter molecule of choice, a suitable 3′ regulatory region, and if desired, a reporter gene, and a marker gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

Once the isolated nucleic acid molecule encoding a methenyltetrahydrofolate synthetase has been cloned into an expression vector, it is ready to be incorporated into a host cell. Recombinant molecules can be introduced into cells, without limitation, via transformation (if the host cell is a prokaryote), transfection (if the host is a eukaryote), transduction (if the host cell is a virus), conjugation, mobilization, or electroporation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, yeast, fungi, mammalian cells, insect cells, plant cells, and the like.

Accordingly, another aspect of the present invention relates to a method of making a recombinant cell. Basically, this method is carried out by transforming a host cell with a nucleic acid construct of the present invention under conditions effective to yield transcription of the DNA molecule in the host cell. Preferably, a nucleic acid construct containing the nucleic acid molecule(s) of the present invention is stably inserted into the genome of the recombinant host cell as a result of the transformation.

Transient expression in protoplasts allows quantitative studies of gene expression since the population of cells is very high (on the order of 106). To deliver DNA inside protoplasts, several methodologies have been proposed, but the most common are electroporation (Neumann et al., “Gene Transfer into Mouse Lyoma Cells by Electroporation in High Electric Fields,” EMBO J. 1: 841-45 (1982); Wong et al., “Electric Field Mediated Gene Transfer,” Biochem Biophys Res Commun 30; 107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81: 7161-65 (1984), which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the gene construct of the present invention into a host cell is fusion of protoplasts with other entities, either minicells, cells, lysosomes, or other fusible lipid-surfaced bodies that contain the chimeric gene (Fraley, et al., Proc. Natl. Acad. Sci. USA, 79:1859-63 (1982), which is hereby incorporated by reference in its entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), which is hereby incorporated by reference in its entirety.

Typically, when a recombinant host is grown for the purpose producing/expressing the desired recombinant protein, an antibiotic or other compound useful for selective growth of the transgenic cells only is added as a supplement to the media. The compound to be used will be dictated by the selectable marker element present in the plasmid with which the host cell was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

An example of a marker suitable for the present invention is the green fluorescent protein (GFP) gene. The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (Prasher et al., “Primary Structure of the Aequorea Victoria Green-Fluorescent Protein,” Gene 111(2):229-233 (1992); U.S. Pat. No. 5,491,084 to Chalfie et al., which are hereby incorporated by reference in their entirety). A plasmid encoding the GFP of Aequorea Victoria is available from the ATCC as Accession No. 75547. Mutated forms of GFP that emit more strongly than the native protein, as well as forms of GFP amenable to stable translation in higher vertebrates, are commercially available from Clontech Laboratories, Inc. (Palo Alto, Calif.) and can be used for the same purpose. The plasmid designated pTα1-GFPh (ATCC Accession No. 98299, which is hereby incorporated by reference in its entirety) includes a humanized form of GFP. Indeed, any nucleic acid molecule encoding a fluorescent form of GFP can be used in accordance with the subject invention. Standard techniques are then used to place the nucleic acid molecule encoding GFP under the control of the chosen cell specific promoter.

The selection marker employed will depend on the target species and/or host or packaging cell lines compatible with a chosen vector.

In carrying out the screening technique of the present invention, test substances which either increase or reduce methenyltetrahydrofolate synthetase expression can be identified.

Another embodiment of the present invention relates to a method of measuring folate status in a sample. This method involves providing a sample and measuring methenyltetrahydrofolate synthetase expression or activity and/or catecholamine activity in the sample. The measured level of methenyltetrahydrofolate synthetase expression or activity and/or catecholamine activity in the sample is correlated to folate status in the sample.

Knowledge of cellular folate concentrations is critical for ensuring disease prevention and for predicting the efficacy and dose of anticancer therapeutic agents. Maintenance of intracellular folate concentrations is critical for maintaining DNA integrity, DNA methylation, and gene expression and therefore disease prevention. Furthermore, the effectiveness of certain antifolate drugs is modified by cellular folate content. Cellular folate levels are determined by dietary folate intake, cellular folate transport and efflux and by rates of folate turnover and/or catabolism. MTHFS accelerates folate catabolism and the expression level of MTHFS (and catacholamines that bind to MTHFS) can be used to predict relative cellular folate concentrations.

In carrying out this aspect of the present invention, the sample is from a subject and can be in the form of a tissue sample or a bodily fluid sample (e.g., blood, serum, or urine).

This aspect of the present invention can be used to treat cancer patients in a variety of ways. In particular, all of the following can be determined based on the folate status of the subject: the cancer state of a subject, the progression of cancer in the subject, and dosing a chemotherapeutic agent. With regard to determining cancer progression based on the folate status, early folate status measurements are compared to subsequent folate status measurements in the subject. By making these measurements, cancer patients can be treated with the amount and type of chemotherapeutic best able to achieve a positive outcome.

This aspect of the present invention can also be used to treat patients with cardiovascular disease. In particular, the cardiovascular disease state of the subject can be determined based on folate status. This is carried out by comparing early folate status measurements to subsequent folate status measurements in the subject. By making these measurements, patients with cardiovascular disease can be treated with the amount and type of therapeutic suitable to obtain the best outcome for the patient.

In carrying out this aspect of the present invention, there are a variety of ways to determine folate status. For example, methenyltetrahydrofolate synthetase expression can be measured in the sample and surrounding normal control tissue. Comparison of MTHFS levels in the sample and normal tissue will enable the determination of relative folate levels. If MTHFS is elevated in the sample, this will indicate that folate levels are depressed in the sample. Alternatively, methenyltetrahydrofolate synthetase activity or catecholamine activity can be measured in the sample and correlated to folate status in the sample. Although not yet verified, MTHFS or catecholamine levels in patient blood and/or serum could be effective approaches as well.

A further embodiment of the present invention relates to a method of treating or preventing cancer in a subject. This method involves administering to the subject a substance which modulates methenyltetrahydrofolate synthetase expression or activity and/or catecholamine activity under conditions effective to treat or prevent cancer in the subject.

In carrying out this aspect of the present invention, cancer can be treated by inhibiting either methenyltetrahydrofolate synthetase expression, methenyltetrahydrofolate synthetase activity, or catecholamine activity. Methenyltetrahydrofolate synthetase activity includes the metabolism of 5-formyltetrahydrofolate and its ability to bind, sequester and channel 10-formyltetrahydrofolate.

5-formyl tetrahydrofolate (“5-formylTHF”) is a natural and endogenous regulator of folate mediated one-carbon metabolism, and that the enzyme MTHFS provides its sole entryway into metabolic pathways. MTHFS regulates folate metabolism by two independent mechanisms: (1) MTHFS is the only enzyme that metabolizes cellular 5-formylTHF, a potent inhibitor of many folate-utilizing enzymes and (2) MTHFS regulates total intracellular folate concentrations (Suh et al., “Purification and Properties of a Folate-Catabolizing Enzyme,” Journal of Biological Chemistry 275:35646-35655 (2000), Anguera et al., “Methenyltetrahydrofolate Synthetase Regulates Folate Turnover and Accumulation,” J. Biol. Chem. 278:29856-29862 (2003), which are hereby incorporated by reference in their entirety). Therefore, MTHFS is a reliable proxy or biomarker for intracellular folate concentrations and also a suitable target for therapeutic anticancer drug development.

About 60 years ago, folates were first isolated and identified to be essential metabolic cofactors for DNA synthesis. Shortly thereafter, inhibitory folate analogs or antifolates were developed which proved to be effective antimicrobial and antineoplasic agents by inhibiting DNA synthesis. These agents include methotrexate, 5-fluoruracil (5-FU) among others, and specifically target folate-dependent enzymes required for nucleotide biosynthesis in cancer cells, because cancer cells display highly elevated expression of folate transport proteins and, therefore, transport antifolate compounds more effectively than normal cells (Zhao et al., Oncogene 22, 7431-57 (2003), which is hereby incorporated by reference in its entirety). The development of antifolates as therapeutic agents remains an active area of research. Because 5-formylTHF is an endogenous and “natural” inhibitor of folate-dependent enzymes and its accumulation in cells occurs at the expense of all other folate cofactors, compounds that mimic 5-formylTHF and block the activity to MTHFS offers a novel anti-cancer therapeutic approach. This approach will simultaneously target all folate-dependent metabolic pathways, and will lead to the development of new pharmaceutical agents (antiproliferative agents). 5-formylTHF is unique from other reduced folate derivatives in that: (1) it is stable towards oxidative degradation; (2) it is not a cofactor for one-carbon transfer reactions; and (3) it is an effective inhibitor of several folate-dependent enzymes in vitro and in vivo, including serine hydroxymethyltransferase (SHMT) (both mitochondrial and cytoplasmic isozymes), AICAR transformylase, the glycine cleavage system, and potentially other folate-dependent enzymes. The regulation of folate metabolism by 5-formylTHF has never been exploited as a target for therapeutic intervention, because little is known about: (1) the regulation of 5-formylTHF concentrations in mammals; (2) the effects of 5-formylTHF on folate-dependent one-carbon metabolic pathways; (3) the consequences of 5-formylTHF accumulation on DNA synthesis and cellular methylation reactions; and (4) the role of methenyltetrahydrofolate synthetase (MTHFS) in regulating 5-formylTHF concentrations. Furthermore, preliminary data indicate that MTHFS expression is elevated in all tumors investigated, suggesting that increased MTHFS activity (resulting in the depletion of the inhibitory folate, 5-formyltetrahydrofolate) may be essential for rapid cell division and perhaps cell viability.

Methenyltetrahydrofolate synthetase (MTHFS) is determined to have another activity in addition to its catalytic activity; it binds tightly 10-formyltetrahydrofolate (10-formylTHF) and its polyglutamate forms. MTHFS binds 10-formylTHF 60-fold more tightly than the substrate 5-formylTHF and is the first identified 10-formylTHF tight binding protein. This 10-formylTHF binding activity of MTHFS governs purine nucleotide biosynthesis by sequestering the cofactor 10-formylTHF, and, therefore, this MTHFS activity is a target for anticancer drug development.

High-affinity MTHFS inhibitors should be N¹⁰-formyl-substituted and be capable of conversion to polyglutamate derivatives in the cell. N⁵-substituted MTHFS inhibitors, including 5-formylTHHF, are not attractive in vivo inhibitors, because they can be slowly metabolized by some mammalian MTHFS enzymes, and because they are not effective substrates for folylpolyglutamate synthetase (FPGS), an enzyme that attaches a polyglutamate chain to folate to help retain folate (or folate inhibitors) in the cell. A number of inhibitors of folate-dependent purine synthesis have been synthesized. 5,10-dideazatetrahydrofolate (DDATHF or Lometrexol) was the first of these drugs to reach clinical trials. DDATHF inhibited mouse enzyme GARFT with a Kᵢ of 6 nM and human GARFT with a Kᵢ of 60 nM. It also proved to be an effective inhibitor of cell growth with an EC₅₀ of 10-30 nM in several different cell lines (McGuire J., “Anticancer Antifolates: Current Status and Future Directions,” Curr Pharm Des. 9(31):2593-613 (2003), which is hereby incorporated by reference in its entirety). A similar compound, 10-formyl-5,10-dideaza-acyclicTHF (10-formyl-DDACTHF) was shown to exhibit some selectively for GARFT. 10-formyl-DDACTHF is a substrate for FPGS and accumulates in cell cultures over 100-fold (Bioorg. Med. Chem. 10(8):2739-49 (2002), which is hereby incorporated by reference in its entirety). The pentaglutamate form effectively inhibits GARFT (Kᵢ=14 nM) and was an effective cytotoxic agent (IC₅₀=60 nM). Given the high affinity of MTHFS for the natural isomer of 10-formylTHF triglutamate (Kᵢ=15 nM) and its relaxed specificity for the pterin moiety, the results of the present work suggest that N¹⁰-substituted analogs of 10-formylTHF may be targeting MTHFS, an effect that may contribute to their cytotoxicity. Furthermore, MTHFS and genetic variants thereof, may also modulate the efficacy of 10-substituted folate analogs that target purine biosynthesis through a channeling mechanism.

Cancers which may be treated or prevented include multiple myeloma, breast cancer, and Wilms tumors.

In practicing the methods of treating or preventing cancer and/or cardiovascular disease in accordance with the present invention, the administering step is carried out by administering to the subject an agent (i.e., a substance which modulates methenyltetrahydrofolate synthetase expression or activity and/or catecholamine activity) orally, intradermally, intramuscularly, intraperitoneally, intravenously, subcutaneously, or intranasally. The agent of the present invention may be administered alone or with suitable pharmaceutical carriers, and can be in solid or liquid form, such as tablets, capsules, powders, solutions, suspensions, or emulsions.

The agent may be orally administered, for example, with an inert diluent, or with an assimilable edible carrier, or it may be enclosed in hard or soft shell capsules, or it may be compressed into tablets, or it may be incorporated directly with food. For oral therapeutic administration, the agent of the present invention may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agent of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

A substance which modulates methenyltetrahydrofolate synthetase expression or activity and/or catecholamine activity may also be administered directly to the airways in the form of an aerosol. For use as aerosols, the substance of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Another aspect of the present invention relates to a method of treating or preventing cardiovascular disease in a subject. This method involves administering to the subject a substance which inhibits methenyltetrahydrofolate synthetase expression or activity and/or catecholamine activity under conditions effective to treat or prevent cardiovascular disease in the subject.

Cardiovascular disease can be treated or prevented by inhibiting either methenyltetrahydrofolate synthetase expression, methenyltetrahydrofolate synthetase activity, or catecholamine activity. In carrying out this aspect of the present invention, the substance can be formulated and administered in substantially the same way as set forth above.

### EXAMPLES

### Example 1

### Materials

Fetal bovine serum, α-minimal essential medium (αMEM), and its α-modification lacking sodium bicarbonate, folate, ribosides, ribotides, deoxyribosides, and deoxyribotides (defined αMEM) were obtained from Hyclone Laboratories. (6S)-5-formylTHF was a generous gift from Eprova. For the isotope tracer studies and folate depletion experiments, fetal bovine serum was dialyzed against ten volumes of phosphate-buffered saline (PBS) at 4° C. for 24 h with buffer changes every 4 h, then charcoal-treated and filtered to deplete serum of folate and other small molecules.

### Example 2

### Sample Collection

Tumor and normal tissue samples were collected at the time of surgery from client-owned dogs and cats presenting for management of cancer at the Cornell University Hospital for Animals. Core tissue samples were removed from the excised tumor and surrounding normal tissue bed using a 4-6 mm punch biopsy after dissection to identify the tumor—normal tissue interface. Normal tissue samples were selected by visual inspection to be 0.5-1.0 cm from the tumor interface and to avoid epidermis and fat. Samples were stored at −80° C. Histopathologic diagnoses were subsequently made for each tumor.

### Example 3

### Tissue Lyses and MTHFS Activity Assays

Tissue samples were sonicated four times for 15 s in a buffer containing 100 mM HEPES pH 7.0, 100 mM sodium chloride, 5 mM EDTA, 1% Tween-20. The solution was clarified by centrifugation. MTHFS activity was determined using a spectrophotometer by monitoring the appearance of 5,10-methenyltetrahydrofolate, which has an absorbance maximum at 355 nm. For a typical assay, 50 μL of clarified tissue supernatant was added to a quartz cuvette containing 100 μM (6S)-5-formylTHF, 1 mM Mg-ATP and 100 mM MES, pH 6.0 and the rate of 5,10-methenylTHF formation was quantified. Activity measurements were normalized to total protein that was quantified using the Lowry-Bensadoun method (Bensadoun et al., “Assay of Proteins in the Presence of Interfering Materials,” Anal Biochem 70:241-250 (1976), which is hereby incorporated by reference in its entirety).

### Example 4

### Western Analyses of HCF and cSHMT

Protein extracts (50 μg/lane) were run on a 12% SDS-PAGE gel, then transferred to a polyvinylidene fluoride microporous membrane (Millipore) using a MiniTransblot apparatus (BioRad). For detection of HCF, the membranes were incubated overnight at 4° C. with primary antisera (1:8,000 dilution) consisting of polyclonal antibodies generated in rabbits. For detection of cSHMT, the membranes were incubated overnight at 4° C. with primary antisera (1:20,000 dilution) consisting of purified polyclonal antibodies generated in sheep that recognize a highly conserved peptide sequence of mouse cSHMT (Liu et al., “Lack of Catalytic Activity of a Murine mRNA Cytoplasmic Serine Hydroxymethyltransferase Splice Variant: Evidence Against Alternative Splicing as a Regulatory Mechanism,” Biochemistry 40:4932-4939 (2001), which is hereby incorporated by reference in its entirety). The membranes were washed with 0.1% Tween-20 in PBS, then incubated for 30 min with either horseradish peroxidase-conjugated rabbit anti-sheep (for cSHMT detection) or goat anti-rabbit (for HCF detection) secondary antibody (1:6500 dilution, Pierce). The membranes were developed using the Super Signal West Pico chemiluminescent detection system (Pierce).

### Example 5

### Folate Depletion of MCF-7 and SH-SY5Y Cells

MCF-7 and SH-SY5Y cells were cultured to 75% confluence in defined αMEM media (lacking folate, ribosides, ribotides, deoxyribosides, and deoxyribotides) for a total of 7 d. Cells were harvested by trypsinization at day 3, 5, 6 and 7, and pelleted by centrifugation. Cell pellets were lysed in a buffer containing 10 mM Tris pH 7.5, 150 mM sodium chloride, 5 mM EDTA, 1% Trition X-100, 20 mM 2-mercaptoethanol, 1 mM phenylmethylsulfonyl fluoride. The solution was centrifuged and the protein concentration of the supernatant was determined. Protein extracts (60 μg/lane) were run on a 12% SDS-PAGE gel, then transferred to a polyvinylidene fluoride microporous membrane (Millipore) using a MiniTransblot apparatus (BioRad). For detection of human MTHFS, the membrane was incubated overnight at 4° C. with primary antisera (1:10,000 dilution) consisting of polyclonal antibodies generated in sheep against a highly conserved peptide sequence of human MTHFS. For detection of human HCF, the membrane was incubated with polyclonal:antibodies (1:7700 dilution) generated in sheep against a conserved sequence of human HCF (Oppenheim et al., “Heavy Chain Ferritin Enhances Serine Hydroxymethyltransferase Expression and De Novo Thymidine Biosynthesis,” Journal of Biological Chemistry 276:19855-19861 (2001), which is hereby incorporated by reference in its entirety). The membranes were washed with 0.1% Tween-20 in PBS, then incubated for 30 min with horseradish peroxidase-conjugated rabbit anti-sheep secondary antibody (1:6500 dilution, Pierce). The membranes were developed using the Super Signal West Pico chemiluminescent detection system (Pierce).

### Example 6

### Stable Isotope Tracer Studies

MCF-7 and SH-SY5Y cells were plated in quadruplicate at 30% confluence in 100 mm Primaria plates (Falcon), and cultured in treatment media, consisting of defined αMEM lacking deoxyribonucleosides, hypoxanthine, and thymidine but supplemented with 0.05 μM folinic acid, 10 μM methionine, 0.2 mM glycine, 1 mg/liter pyridoxine, 250 μM formate, 26 mg/liter L-[5,5,5-²H₃]leucine, and 250 μM L-[2,3,3-²H₃]serine. Cells were cultured with treatment media for a total of 8 d, and the medium was replaced every 2 d. The cells were harvested by washing with ice-cold PBS, then trypsinized and pelleted by centrifugation. Cellular protein and genomic DNA were isolated and analyzed as described previously (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety).

### Example 7

### Analyses of MTHFS, HCF, and cSHMT Levels in Tumors

There was an increase in the specific activity of MTHFS in tumors relative to surrounding normal tissues for all 14 sets of paired tumor and normal tissue samples (Table 1).

TABLE 1
MTHFS activity in normal and tumor tissue biopsies.

| | | Normal Tissue | Tumor Tissue | Fold |
|---|---|---|---|---|
| Species | Tumor type | Specific Activity | Specific Activity | Increase |
| canine | mastocytoma | 0.07 ± 0.03 | 0.93 ± 0.01 | 13.30 |
| canine | mastocytoma | 0.08 ± 0.02 | 1.97 ± 0.08 | 24.63 |
| canine | mastocytoma | 0.18 ± 0.01 | 1.77 ± 0.09 | 12.64 |
| canine | mastocytoma | 0.33 ± 0.10 | 1.42 ± 0.04 | 4.30 |
| canine | mastocytoma | 0.06 ± 0.01 | 0.30 ± 0.01 | 5.00 |
| canine | sarcoma | 1.83 ± 0.08 | 3.38 ± 0.10 | 1.85 |
| canine | sarcoma | 0.36 ± 0.07 | 2.02 ± 0.02 | 5.61 |
| canine | sarcoma | 0.22 ± 0.01 | 1.47 ± 0.26 | 6.68 |
| feline | sarcoma | 0.09 ± 0.05 | 1.35 ± 0.04 | 15.00 |
| canine | melanoma | 1.43 ± 0.05 | 1.86 ± 0.09 | 1.31 |
| canine | osteosarcoma | 0.20 ± 0.03 | 1.29 ± 0.06 | 6.45 |
| canine | rectal | n.d. | 0.03 ± 0.001 | \ |
| | carcinoma | | | |
| *feline | hemangio- | 0.57 ± 0.03 | 1.72 ± 0.13 | 3.02 |
| | sarcoma | | | |
| *feline | hemangio- | 0.49 ± 0.04 | 3.43 ± 0.10 | 7.00 |
| | sarcoma | | | |
| feline | breast | n.d. | 0.12 ± 0.01 | \ |

n.d.-MTHFS activity in the tissue sample was below background (not detected).
The specific activities of MTHFS (in units of pmoles 5,10-methenylTHF/min/μg protein) were determined for the feline and canine tissue samples. Specific activity values shown represent the average of triplicate measurements and variation is expressed as standard deviations of the mean. The * indicates two independent biopsies from the same animal.

For the normal tissue samples, the specific activity values ranged from undetectable to 1.83 pmoles 5,10-methenylTHF/min/μg protein, and for tumor tissues the range was 0.03 to 3.4 pmoles 5,10-methenylTHF/min/μg protein (Table 1). The average MTHFS activity in all 14 sets of tumor samples was 3.4-fold higher than the normal tissues, and the means were statistically different by one-way ANOVA analysis, yielding a P-value of 0.003 (Table 2).

TABLE 2
Statistical analyses of MTHFS specific
activity in the animal tumor samples.

| Samples | Normal | Tumor | P-value |
|---|---|---|---|
| All animal tumors | 0.45 ± 0.58 | 1.46 ± 0.93 | 0.003 |

Canine

| | | | |
|---|---|---|---|
| All samples | 0.48 ± 0.62 | 1.50 ± 0.91 | 0.008 |
| Mastocytomas | 0.10 ± 0.056 | 1.28 ± 0.67 | 0.017 |

Feline

| | | | |
|---|---|---|---|
| All samples | 0.31 ± 0.31 | 1.35 ± 1.23 | 0.180 |

The average specific activity values for 14 sets of normal and tumor tissues were analyzed. Student's t-tests were performed assuming equal variance, except for the canine mastocytoma samples, which were performed assuming unequal variance. Due to small sample size, feline samples were compared using the non-parametric median test.

Similar trends were observed for the individual canine and feline tumor sets (Table 2). However, the means were not significantly different for feline samples because of insufficient sample size (Table 2). Previous work found that small elevations (1.8-fold) in MTHFS activity lead to a 40% decrease in total intracellular folate in MCF-7 cells (Anguera et al., “Methenyltetrahydrofolate Synthetase Regulates Folate Turnover and Accumulation,” J. Biol. Chem. 278:29856-29862 (2003), which is hereby incorporated by reference in its entirety), indicating that the increased rates of folate catabolism and folate deficiency observed in tumors may result from increased MTHFS activity.

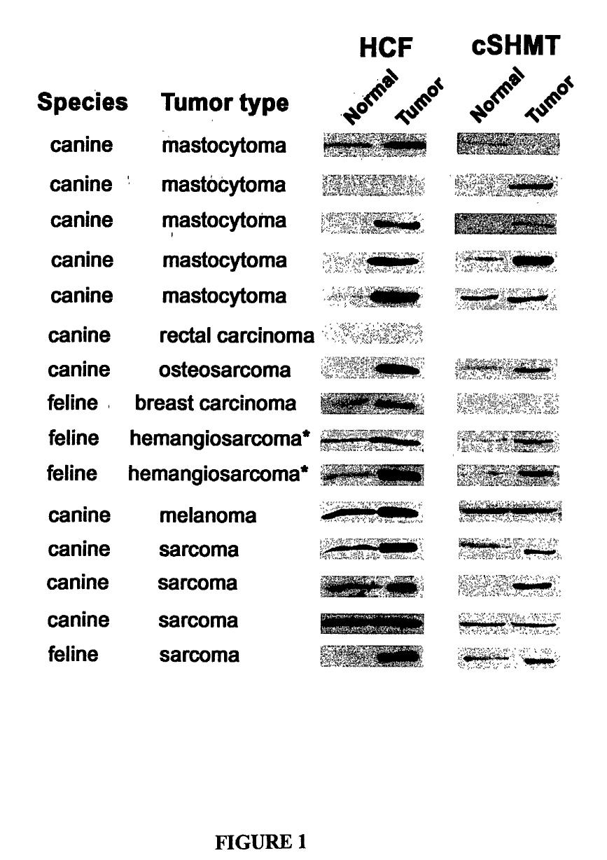

HCF also accelerates folate catabolism in cell culture models (Suh et al., “Purification and Properties of a Folate-Catabolizing Enzyme,” Journal of Biological Chemistry 275:35646-35655 (2000), which is hereby incorporated by reference in its entirety). Thus, HCF protein levels were examined by Western Blotting using polyclonal antibodies that specifically recognize the heavy-chain monomer of ferritin. HCF protein levels were increased in 11 out of the 14 sets of tumor samples relative to the paired normal tissue (FIG. 1). For two sets of samples, mastocytoma and rectal carcinoma, HCF protein was not detected, and in one sarcoma sample, HCF levels were the same in both the normal and tumor tissue (FIG. 1). The increased expression of HCF and MTHFS in tumors provides a mechanism for folate deficiency and DNA hypomethylation that is observed in cancer, because both proteins accelerate folate turnover.

The cSHMT enzyme, although not expressed in all cells (Girgis et al., “Molecular Cloning, Characterization and Alternative Splicing of the Human Cytoplasmic Serine Hydroxymethyltransferase Gene,” Gene 210:315-324 (1998), which is hereby incorporated by reference in its entirety), is a metabolic switch that directs one-carbon units to dTMP biosynthesis and inhibits SAM biosynthesis (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). Therefore, cSHMT may contribute to DNA hypomethylation that occurs early in cellular transformation. Although other studies have observed elevated SHMT activity in tumors (Snell et al., “Enzymic Imbalance in Serine Metaoblism in Human Colon Carcinoma and Rat Sarcoma,” British Journal of Cancer 57:87-90 (1988), which is hereby incorporated by reference in its entirety), these studies never distinguished between the contribution of the mitochondrial and cytoplasmic isozymes. Western analyses were performed on the 14 tumor sets using polyclonal antibodies that recognize the mouse form of cSHMT (Liu et al., “Lack of Catalytic Activity of a Murine mRNA Cytoplasmic Serine Hydroxymethyltransferase Splice Variant Evidence Against Alternative Splicing as a Regulatory Mechanism,” Biochemistry 40:4932-4939 (2001), which is hereby incorporated by reference in its entirety). Eight tumor samples had increased levels of cSHMT protein relative to normal tissue (FIG. 1). There was no change in cSHMT protein levels in three sets of the tumor and normal tissue samples, which included mastocytoma, melanoma, and sarcoma tumor types (FIG. 1). One of the mastocytoma samples displayed less cSHMT protein relative to the normal tissue, and no cSHMT protein was detected in the rectal carcinoma or the breast carcinoma samples. This is the first study to determine specifically the levels of cSHMT in tumor samples, and to demonstrate that cSHMT expression is elevated in the majority of cancers studied here.

### Example 8

### Impact of Increased MTHFS Activity on the Thymidylate Biosynthetic Pathway

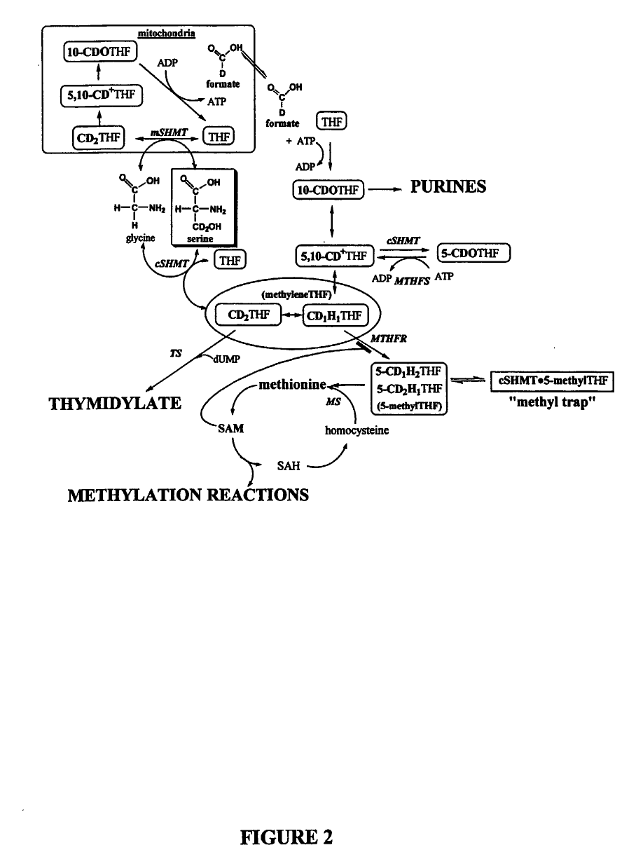

Previously, applicant demonstrated that the cSHMT enzyme is a metabolic switch that directs one-carbon units to dTMP synthesis and inhibits SAM biosynthesis (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). The SHMT enzyme catalyzes the reversible transfer of the hydroxymethyl group of serine to THF generating methyleneTHF and glycine (Stover et al., “Serine Hydroxymethyltransferase Catalyzes the Hydrolysis of 5,10-Methenyltetrahydrofolate to 5-Formyltetrahydrofolate,” Journal of Biological Chemistry 265:14227-14233 (1990), which is hereby incorporated by reference in its entirety). This reaction is inhibited by 5-formylTHF. In addition to accelerating folate turnover, MTHFS catalyzes the ATP-dependent conversion of 5-formylTHF to 5,10-methenylTHF (FIG. 2). 5-formylTHF is the only folate derivative that is not a cofactor for one-carbon transfer reactions but is a potent inhibitor of folate dependent enzymes including cSHMT (Stover et al., “The Metabolic Role of Leucovorin,” Trends in Biochemical Sciences 18:102-106 (1993), which is hereby incorporated by reference in its entirety). MTHFS participates in a futile cycle with cSHMT, because cSHMT has a second catalytic activity, the conversion of 5,10-methenylTHF to 5-formylTHF (Stover et al., “The Metabolic Role of Leucovorin,” Trends in Biochemical Sciences 18:102-106 (1993), which is hereby incorporated by reference in its entirety). Once formed, 5-formylTHF remains bound to cSHMT as a slow-releasing inhibitor (Stover et al., “5-Formyltetrahydrofolate Polyglutamates Are Slow Tight Binding Inhibitors of Serine Hydroxymethyltransferase,” Journal of Biological Chemistry 266:1543-1550 (1991), which is hereby incorporated by reference in its entirety). Because all of the tumor biopsies displayed elevations in MTHFS activity and changes in MTHFS activity influence cSHMT activity (Girgis et al., “5-Formyltetrahydrofolate Regulates Homocysteine Remethylation in Human Neuroblastoma,” Journal of Biological Chemistry 272:4729-4734 (1997), which is hereby incorporated by reference in its entirety), applicant determined the effects of increased MTHFS activity on the flux of one-carbon units into the dTMP and methionine pathways originating from the cSHMT in MCF-7 and SH-SY5Y neuroblastoma cell lines transfected with human MTHFS cDNA (Anguera et al., “Methenyltetrahydrofolate Synthetase Regulates Folate Turnover and Accumulation,” J. Biol. Chem. 278:29856-29862 (2003), which is hereby incorporated by reference in its entirety). Both dTMP and methionine biosynthesis compete for a limited pool of methyleneTHF cofactors, as shown in FIG. 2 (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). The one-carbon unit of methyleneTHF is derived primarily from serine, and the hydroxymethyl group of serine can be directly incorporated into methyleneTHF through the catalytic activity of cSHMT. Alternatively, the hydroxymethyl group of serine can be converted to formate in the mitochondria, and enter the cytoplasmic folate pool in the form of 10-formylTHF which can be reduced to methyleneTHF (FIG. 2). When cells are labeled with L-[2,3,3-²H₃]serine, two labeled forms of methyleneTHF are generated. MethyleneTHF that is synthesized from serine in the cytoplasm will contain 2 deuterium atoms (CD2), whereas methyleneTHF that is generated from mitochondrial conversion of serine to formate will contain 1 deuterium atom (CD1). Therefore, the ratio of CD2/CD1 in cellular protein (corresponding to labeled methionine) and genomic DNA (corresponding to dTMP) can be used to measure the efficiency of the cSHMT enzyme. Previously, applicant used this method to determine that cSHMT preferentially generates methyleneTHF for dTMP biosynthesis, indicating that methyleneTHF generated from cSHMT is not in complete equilibrium with methyleneTHF generated from formate (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). These results indicated that cSHMT may be compartmentalized or may physically interact with thymidylate synthetase (TS) (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety).

To determine the effects of increased MTHFS activity (and, therefore, 5-formylTHF depletion) on cSHMT activity, cell lines with increased MTHFS activity were cultured with L-[2,3,3-²H₃]serine, and the ratio of CD1 and CD2 label incorporated into dTMP and methionine was determined as described previously (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). Both MCF-7 and SH-SY5Y cells display increased incorporation of CD2 into dTMP compared to methionine (Table 3), consistent with previous experiments, demonstrating that cSHMT preferentially directs one-carbon units for dTMP biosynthesis over methionine synthesis (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety). However, cell lines with increased MTHFS activity no longer exhibit preferential enrichment of cytoplasmic-derived methyleneTHF into dTMP as determined by the ratio of CD2 to CD1 in dTMP and methionine (Table 3).

TABLE 3
The effect of increased MTHFS expression on 3-[²H]serine
metabolism in MCF-7 and SH-SY5Y cells.

| Cell Line | Met | dT | DHA |
|---|---|---|---|
| MCF-7 | 9.4 | 13.8 | 66.6 |
| MCFMTHFS | 9.0 | 8.9 | 64.2 |
| SH-SY5Y | 7.1 | 11.3 | 80.1 |
| 5YMTHFS | 8.0 | 9.0 | 81.4 |

Cellular protein and DNA were isolated from cells cultured for eight days in defined medium containing L-[5,5,5-²H₃]leucine and L-[2,3,3-²H₃]serine. Isotopic enrichments of one carbon transferred from L-[2,3,3-²H₃]serine into methionine and thymidine (dT) were determined by analysis of the cellular protein and genomic DNA pools, respectively. The enrichment of the 3-carbon of serine into the cellular serine pool was determined from serine isolated from the protein pool, after its conversion to dehydroalanine (DHA). All values are expressed as the ratio of carbons containing two deuterium atoms in the target compound divided by the total number of carbons that contain one or two deuterium atoms.

This loss in the enrichment of cSHMT-derived methyleneTHF into dTMP can be explained by the observation that SH-SY5Y cells with increased MTHFS activity have dramatically elevated levels of the TS protein. Because cSHMT is limiting for dTMP synthesis in MCF-7 and SH-SY5Y cells (Herbig et al., “Cytoplasmic Serine Hydroxymethyltransferase Mediates Competition Between Folate-Dependent Deoxyribonucleotide and S-Adenosylmethionine Biosyntheses,” Journal of Biological Chemistry 277:38381-38389 (2002), which is hereby incorporated by reference in its entirety), increases in TS levels in the absence of corresponding increases in cSHMT protein levels result in TS acquiring methyleneTHF from the formate pathway (FIG. 2), thereby diluting the contribution of cSHMT to dTMP biosynthesis. These results support previous suggestions that cSHMT may physically interact with TS and direct methyleneTHF to this pathway. Therefore, it is concluded that activation of MTHFS expression disrupts the ability of cSHMT to preferentially shuttle serine-derived one-carbon units into dTMP biosynthesis, resulting in near-equal incorporation of label into both dTMP and methionine.

### Example 9

### Effect of Folate Depletion on MTHFS and HCF Expression in Cell Cultures

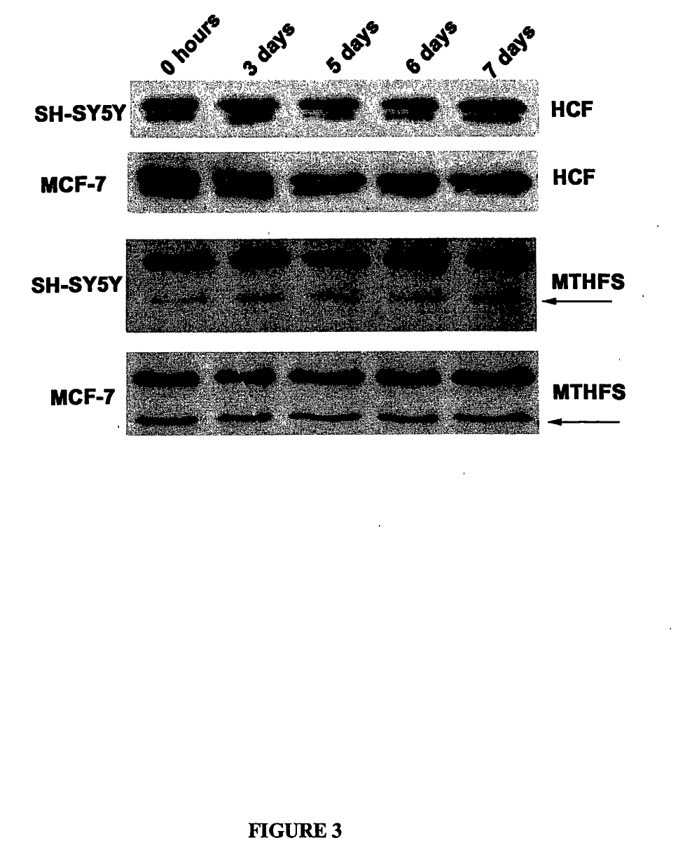

Expression of the folate receptor protein responds to cellular folate status, with increased expression of the receptor occurring when cells become folate deficient (Sadasivan et al., “The Half-Life of the Transcript Encoding the Folate Receptor A in KB Cells is Reduced By Cytosolic Proteins Expressed in Folate-Replete and Not in Folate-Depleted Cells,” Gene 291:149-158 (2002); Mendelsohn et al., “The Role of Dietary Folate in Modulation of Folate Receptor Expression, Folylpolyglutamate Synthetase Activity and the Efficacy and Toxicity of Lometrexol,” Advances in Enzyme Regulation 36:365-381 (1996), which are hereby incorporated by reference in their entirety). To determine if folate deficiency influences the expression of the proteins that degrade folate, the protein levels of MTHFS and HCF were determined in MCF-7 and SH-SY5Y cells cultured in folate-deficient medium. Cells were cultured in defined medium lacking folate for a total of seven days, and cells were harvested at defined time intervals. The levels of MTHFS and HCF proteins present in cellular lysates were determined by Western Analyses, and the results indicate that the expression of these proteins did not change significantly over time in either cell line (FIG. 3). These results indicate that enzyme-mediated folate catabolism does not respond to cellular folate deficiency in cell cultures.

DNA hypomethylation is an early event in cellular transformation, and numerous studies have demonstrated loss of methylation during folate deficiency. Because AdoMet synthesis is dependent on the availability of folate-activated one-carbon units, folate deficiency may contribute to genomic instability and alterations in gene expression that are associated with cellular transformation. Alterations in folate metabolism compromise the supply of AdoMet for DNA methylation, and applicant's recent work has shown that elevated rates of folate catabolism result in folate deficiency and disruption of folate metabolism (Anguera et al., “Methenyltetrahydrofolate Synthetase Regulates Folate Turnover and Accumulation,” J. Biol. Chem. 278:29856-29862 (2003); Girgis et al., “5-Formyltetrahydrofolate Regulates Homocysteine Remethylation in Human Neuroblastoma,” Journal of Biological Chemistry 272:4729-4734 (1997), which are hereby incorporated by reference in their entirety). Therefore, increased expression of MTHFS and HCF may provide the first mechanism that accounts for the folate deficiency in cancer. In order to test this hypothesis, the levels of these proteins were determined in 14 matched sets of tumor biopsies and corresponding normal tissue samples. This is the first study to determine the activity of MTHFS in tumor tissue, and MTHFS activity was found to be increased between 1.3 to 24-fold in all 14 sample sets. The melanoma sample had the lowest fold-increase in MTHFS activity (1.3-fold) and the normal tissue sample may have had invasive melanoma extending into apparently normal neighboring tissue. HCF has previously been shown to be elevated in various cancer models, and the results from this study are in agreement with previous work (Suh et al., “New Perspectives On Folate Catabolism,” Annu Rev Nutr 21:255-282 (2001), which is hereby incorporated by reference in its entirety). The supply of AdoMet required for DNA methylation is also influenced by cSHMT, which acts as a metabolic switch that preferentially directs one-carbon units to dTMP over methionine biosynthesis. This study, the first to determine the levels of cSHMT present in tumor samples, identified increased expression of cSHMT in 8 tumor samples relative to adjacent normal tissue. The expression of cSHMT was elevated in 3 of the 4 sarcoma tumor samples, and these tumors also displayed increased expression of HCF. It is not clear why cSHMT was not detected in either the feline breast cancer tumor and normal breast tissue sample, because it has been detected in the MCF-7 cell line that is derived from a human mammary adenocarcinoma (Oppenheim et al., “Mimosine Is a Cell-Specific Antagonist of Folate Metabolism,” Journal of Biological Chemistry 275:19268-19274 (2000), which is hereby incorporated by reference in its entirety).

MTHFS and cSHMT enzymes participate in a metabolic futile cycle. Therefore, the effects of increased MTHFS activity on cSHMT activity were determined using cell culture models. In cells with increased MTHFS activity, methyleneTHF derived from cSHMT is no longer preferentially directed to dTMP synthesis. Because MTHFS activity was increased in all tumors in this study, applicant predicts that these tumors display both folate deficiency and altered cSHMT function. The levels of folate and AdoMet were not determined in this study because of lack of sufficient sample material. Therefore, additional studies are needed to confirm the impact of altered MTHFS activity on folate metabolism, cellular folate and AdoMet concentrations.

The results presented in this study strongly indicate that MTHFS may serve as a biomarker for cellular transformation. The samples examined in this study represent spontaneously arising neoplasms in an advanced, invasive clinical stage. Surgery was conducted with curative intent or as a planned component of multimodality therapy. Additional studies are currently underway to determine when MTHFS expression is induced during the stages of cellular transformation and whether observations made in this study are specific to malignancy or occur in benign alterations such as inflammatory or infectious conditions as well.

The data presented in this paper suggest two possible mechanisms for the impact of MTHFS on folate deficiency observed in cancer models. One possible mechanism is that increased expression and activity of MTHFS results in cellular folate deficiency, which compromises the supply of one-carbon units needed to synthesize AdoMet, resulting in decreased AdoMet availability for DNA methylation. This mechanism requires that elevations of MTHFS activity occur prior to the loss of methylation observed in the early transformation of normal epithelium to early adenoma. Alternatively, loss of methylation causes increased expression of MTHFS (it is unknown whether the MTHFS promoter is affected by methylation status), and the increase in MTHFS activity generates folate deficiency once cellular transformation has already begun. Additional studies are necessary to distinguish between these two mechanisms. In conclusion, the work presented here indicates that alterations in MTHFS activity may have diagnostic value in serving as an indicator of both tumor folate status and antifolate efficacy, as well as a marker of neoplastic transformation.

### Example 10

### Materials

(6S)-[3′,5′,7,9⁻³H]5-formylTHF (40 Ci/mmol) and (6S)-[3′,5′,7,9⁻³H]-5-methylTHF (30 Ci/mmol) were obtained from Moravek Biochemicals, Inc. (6R,6S)-5-formylTHF was from SAPEC, and (6S)-5-methylTHF and (6S)-5-formylTHF were the generous gift of Eprova AG. HEPES, ATP, Tris, sodium periodate, dopamine, epinephrine, L-dopa, and N-acetyldopamine (NADA) were purchased from Sigma. Oxidized NADA was prepared by dissolving 5 mg NADA in 20 mM hydrochloric acid and oxidized with 5 mg of sodium periodate and diluted 1:1 with 1 M Tris pH 8.0. Fetal bovine serum, α-minimal essential medium (αMEM), and α-modification lacking sodium bicarbonate, folate, ribosides, ribotides, deoxyribosides, and deoxyribotides (defined αMEM) were obtained from Hyclone Laboratories.

### Example 11

### Cell Lines and Medium

The human MCF-7 mammary adenocarcinoma cells (ATCC catalog number HTB22) have been described elsewhere (Girgis et al., J Biol Chem 272:4729-4734 (1997), which is hereby incorporated by reference in its entirety). For folate turnover studies, fetal bovine serum was dialyzed against ten volumes of phosphate-buffered saline (PBS) at 4° C. for 24 h with buffer changes every 4 h to deplete serum of folate and other small molecules. The serum was then charcoal-treated to remove any remaining folate.

### Example 12

### Purification of Recombinant Mouse MTHFS

The mouse MTHFS cDNA was isolated from total liver cDNA and cloned into a pet28a expression vector. E. coli BL21 Star cells (Invitrogen) were transformed with this MTHFS expression vector, and the recombinant mouse MTHFS protein was purified using metal affinity chromatography.

### Example 13

### Inductively Coupled Plasma Mass Spectrometry (ICP-MS) of Recombinant Mouse MTHFS Protein

A 200 μM sample of recombinant mouse MTHFS protein was dialyzed against 2 L of 100 mM Tris pH 8.0 at 4° C. overnight with two buffer changes. The protein sample and samples of the dialysis buffer were analyzed by ICP-MS at the Plant Sciences Department at Cornell University.

### Example 14

### Removal of the Chromophore from Recombinant Mouse MTHFS Protein

The chromophore was removed from recombinant mouse MTHFS protein by two different procedures: application of the protein to a 5-formylTHF-sepharose column or by solvent extraction. The chromophore was removed from the protein by loading recombinant mouse MTHFS protein onto a 5-formylTHF-sepharose column equilibrated with 20 mM Tris pH 7.0 and 20 mM 2-mercaptoethanol. The column was washed with water and the chromophore was collected in the flow-through fractions. The chromophore was also removed from the protein by extraction with 1:3 dilution of 100% acetonitrile/water. The protein solution was heated to 95° C. for 15 min, followed by incubation on ice for 3 h. Glass wool was used to separate the precipitated protein from the solvent containing the chromophore. For the analysis using electrospray ionization mass spectrometry, the chromophore was removed from the protein either using solvent extraction or with the 5-formylTHF-sepharose column. Flowthrough fractions from the 5-formylTHF-sepharose column were collected and concentrated under vacuum. The dried pellet was resuspended in 100% acetic acid, and analyzed by ESI-MS. Oxidized NADA standards were prepared in the same buffer as the chromophore and concentrated under vacuum and analyzed by ESI-MS.

### Example 15

### Reconstitution of Purified Rabbit Liver MTHFS with NADA

NADA solutions were prepared by dissolving 3 mg NADA in 1 ml of 20 mM hydrochloric acid (HCl), and the acid neutralized by the addition of an equal volume of 1 M potassium phosphate pH 7.2. For the oxidized NADA sample, 3 mg of sodium periodate (Sigma) were added to the NADA solution, then an equal volume of 1 M potassium phosphate pH 7.2 was added to adjust the pH to 7.0. NADA, oxidized NADA, and a buffer control (100 μL of 1 M potassium phosphate pH 7.2) were added to purified rabbit liver MTHFS (0.025 μmoles) and yeast aldehyde dehydrogenase (10 U/sample) and incubated at room temperature for 15 min. The samples were transferred to dialysis tubing (molecular weight cut off=5000 Da) and dialyzed overnight against 4° C. in 2 L of 5 mM potassium phosphate pH 7.2 with two buffer changes. The next day the proteins were clarified by centrifugation, and a UV spectrum was recorded for each sample.

### Example 16

### Growth of Recombinant Mouse MTHFS in M9 Minimal Media

BL21 star cells transformed with the mouse MTHFS cDNA in the pET-28a vector were used to inoculate 5 mL starter cultures of LB containing 50 μg/ml kanamycin. The cultures were grown for 16 h at 37° C., and an aliquot was used to inoculate a 50 ml LB culture with 50 μg/ml kanamycin. The cultures were incubated at 37° C. until A₅₅₀ₙₘ=0.7, the cells were pelleted by centrifugation, and used to inoculate 1 L of minimal media containing 2×M9 salts, 0.2 mM magnesium sulfate, 0.4% glucose, 12.5 mg ferrous iron sulfate, 1× vitamins solution (Gibco), 40 mg of each of the 20 biologically-relevant amino acids (Sigma), and 50 μg/mL kanamycin. The cultures were incubated at 37° C. until A₅₅₀ₙₘ=0.7, then 1.5 mM IPTG and 10 mg NADA (dissolved in ethanol/water) were added. The cultures were incubated at 25° C. for 5 hours. Cells were harvested by centrifugation, and stored at −80° C.

### Example 17

### MALDI-TOF and ESI-MS Experiments

Spectra were obtained with a Bruker Esquire-LC (Bremen, Germany) ion trap mass spectrometer using both positive and negative ion modes. Samples were infused into the source using a Cole-Parmer 74900 series infusion pump at a rate of 1 μL/min through a fused silica line, with methanol or methanol/water (1/1, v/v) as infusion solvents. The drying temperature (heated capillary) was maintained at 225° C. using N₂ gas at 4 L/min and nebulizer flow at 16 psi. The HV capillary was 4000 V (−4000 for negative ion spectra), the cap exit offset 78.6 V, with a 55 V difference between Lens 1 and Lens 2, and a 40 V difference between the two skimmers. MS spectra were obtained by averaging 40 spectra from m/z 95-850. Ten to twelve scans were averaged for MS/MS and MSn spectra using a fragmentation rF amplitude between 0.8 and 1.4 and an isolation width of +/−1.5 u. Unit resolution was obtained for all spectra. Data were processed using the Bruker DataAnalysis software, and all MS analyses were performed at the BioResource Center of Cornell University.

### Example 18

### HPLC Analyses of In-Vitro MTHFS-Mediated Folate Catabolism Reactions

Recombinant mouse MTHFS protein (50 μM) was incubated with 50 μM CuCl₂, 50 μM NADA (oxidized with equimolar amounts of sodium periodate, extracted with ethyl acetate, and diluted 1:1 with 1 M Tris pH 8.0), in 50 mM Tris pH 8.0 for 5 min, then dialyzed against 2 L of 50 mM Tris pH 8.0 at 4° C. for 5 hours to remove unbound CuCl₂ and oxidized NADA. (6S)-[³H]-5-methylTHF or (6S)-[³H]-5-formylTHF, were evaporated to dryness using a speedvac, resuspended in 20 μL of 50 μM unlabeled (6S)-5-methylTHF or (6S)-5-formylTHF in 100 mM MES pH 6.5, then approximately 275,000 cpm of the labeled folate was added immediately to either the dialyzed mouse MTHFS protein or the dialysis buffer as a control. The mixture was incubated 5 min at room temperature, then either analyzed immediately by HPLC or flash frozen using dry ice/ethanol. The reactions were spiked with a mixture of unlabeled pABG, (6R,S)-5-formylTHF, and (6S)-5-methylTHF, then analyzed on a Shimadzu HPLC equipped with a diode array UV spectrophotometric detector and a Luna 5μ 250×4.6 mm C18 column (Phenomenex). A binary buffer gradient (Anguera et al., J. Biol. Chem. 278:29856-29862 (2003), which is hereby incorporated by reference in its entirety) was used to separate folate degradation products, intact folate, and oxidized NADA. For each analysis, 1.0 ml fractions were collected into scintillation tubes, and the tritium was quantified using a Beckman LS 6500 liquid scintillation counter. Radioactive peaks were identified by their elution times relative to internal standards.

### Example 19

### Determination of Folate Turnover in Cultured Cells

Cell monolayers at 75% confluence were washed with PBS, then labeled for 12 h in defined αMEM lacking folate and glycine and supplemented with 25 nM (6S)-[³H]5-formylTHF. For the chase, cell monolayers were washed with 10 ml of PBS, trypsinized, and pelleted by centrifugation. The cells were seeded in triplicate (1×10⁶ cells) into 100 mm culture plates containing 10 ml of defined αMEM supplemented with 2 μM folic acid. Catecholamines (20 μM dopamine, NADA, and L-dopa) were prepared freshly and added to the chase media prior to the addition of labeled cells. Cells were harvested at defined time points, the medium was removed and the tritium in the medium was quantified. The cell monolayers were washed with PBS and lysed with 0.2 M ammonium hydroxide. Tritium remaining in the cells was quantified using a Beckman LS 8100 liquid scintillation counter.

### Example 20

### Determination of Radiolabeled Folate Accumulation in Cultured Cells Treated with Catecholamines

Cell monolayers at 70% confluence in 6-well plates were labeled for 12 h in defined αMEM lacking folate and glycine, but supplemented with 25 nM (6S)-[³H]5-formylTHF and 0, 25 μM, 50 μM, 75 μM, 100 μM, or 150 μM NADA, dopamine, or epinephrine. Cells were harvested by washing with 5 mL PBS, trypsinized, and pelleted by centrifugation and viable cells (determined by their ability to exclude trypan blue) were quantified. The cell pellets were lysed with 0.2 M ammonium hydroxide, and the intracellular tritium was quantified using a Beckman LS 8100 liquid scintillation counter.

### Example 21

### Identification of a Small Molecule Bound to Recombinant Mouse MTHFS Protein

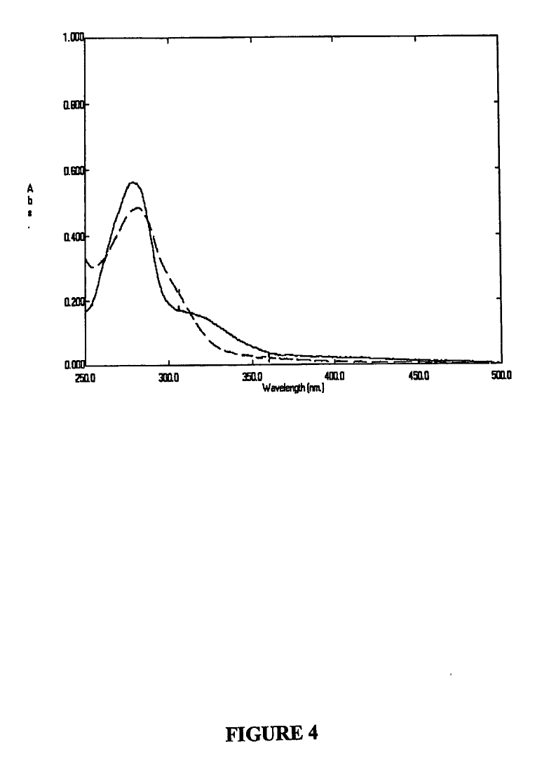

The murine MTHFS cDNA was cloned in frame with a hexa-histidine tag, expressed in E. coli, and purified using metal affinity chromatography. The absorbance spectrum of the purified, dialyzed recombinant mouse MTHFS protein indicates the presence of an absorption shoulder at 320 nm that is absent in the purified rabbit liver MTHFS protein, indicating that a chromophore is bound to the protein (FIG. 4). Removal of the hexa-histidine tag from recombinant mouse MTHFS using thrombin treatment did not change the UV spectrum of the protein. The UV spectrum of the purified rabbit liver MTHFS protein lacks the shoulder peak at 320 nm (FIG. 4), but does display a less-pronounced absorbance shoulder at 305 nm which was identified by HPLC to be tightly bound 5-formylTHF that does not dissociate during dialysis.

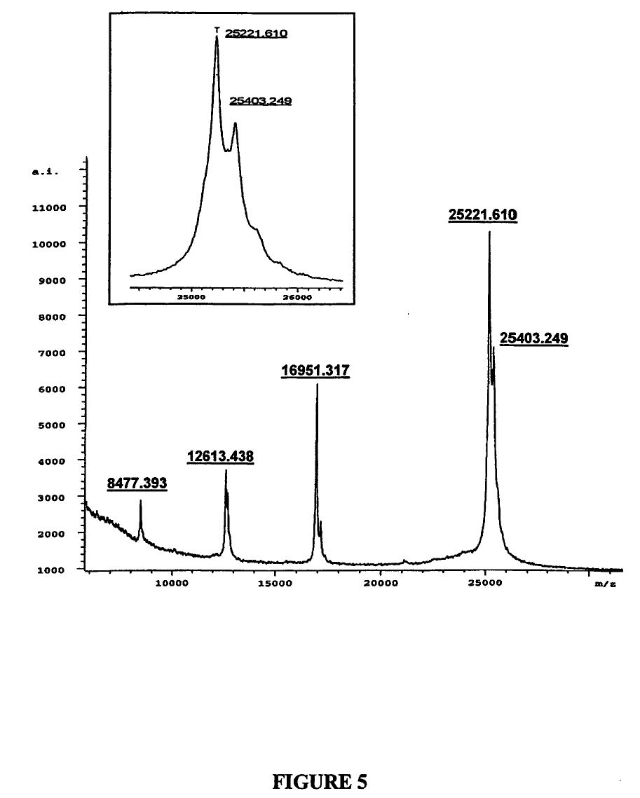

The mass of the recombinant MTHFS protein was determined by MALDI-TOF mass spectrometry (FIG. 5). The mass spectrum of recombinant mouse MTHFS protein includes a peak with a m/z=16951.317, representing horse apomyoglobin that was added as an internal standard. The recombinant mouse MTHFS protein exhibits two peaks with masses of 25221.610 and 25403.249 Da, and there are two peaks corresponding to the multiply charged ions (M+2) at m/z=12613.438. The theoretical molecular mass of mouse MTHFS with the hexa-histidine tag was calculated to be 25208.79 Da and analysis by SDS-PAGE was in agreement with this value. The peak observed at 25221.610 Da for the recombinant mouse MTHFS protein is similar to the estimated molecular mass of 25208.79, whereas the peak at 25403.249 Da corresponds to the mass of MTHFS plus 182 Da, which represents the presence of the bound chromophore observed in the UV spectral studies. MALDI-TOF analysis was repeated three times using recombinant mouse MTHFS protein from different purifications, and the mass differences among the peaks representing halo-MTHFS and apo-MTHFS varied between 179-182 Da. A sample of recombinant mouse MTHFS protein with salts and contaminants removed using C-18 Zip-Tips yielded a mass difference of 190 Da. Examination of the MALDI-TOF spectrum indicates that most of the protein lacks the bound compound (FIG. 5 inset). There are other smaller peaks of greater molecular mass in addition to the two predominant two peaks of m/z=25221.610 and 25403.249 (FIG. 5). The differences in the masses of the chromophore could signify the presence of more than one compound bound to the protein, or that the chromophore is unstable during the sample preparation and analysis.

The recombinant mouse MTHFS protein was also analyzed for metal content using inductively coupled plasma mass spectrometry (ICP-MS). Because MTHFS is known to bind Mg-ATP (Jolivet et al., Oncologist 1:248-254 (1996); Dayan et al., Gene 165:307-311 (1995); Bertrand et al., Biochimica et Biophysica Acta 1266:245-249 (1995), which are incorporated by reference in their entirety), there may be a metal binding site on the protein that can accommodate metal ions for electrons generated in folate catabolism reaction. Following extensive dialysis, the protein sample was found to contain elevated concentrations of copper II (Cu²⁺) relative to the dialysis buffer (Table 4), indicating that about 18% of the MTHFS protein contained bound copper following purification of the MTHFS protein on a metal affinity column and extensive dialysis.

TABLE 4
ICP-MS analysis of recombinant mouse MTHFS protein.

| ELEMENT | PPM | CONCENTRATION (μM) |
|---|---|---|
| Cu | 0.2249 | 35.51 |
| Zn | 3.2281 | 49.36 |
| Mg | 0.8631 | 3.54 |
| Ni | 0.556 | 9.47 |
| Na | 91.468 | 3980 |
| S | 26.134 | 816.6 |
| P | 7.3947 | 238.9 |

The metal content present in a sample of recombinant mouse MTHFS (200 μM) was determined using ICP-MS. Recombinant mouse MTHFS protein was dialyzed against 2 L of 100 mM Tris pH 8.0 overnight at 4° C. with two buffer changes. Both the protein and dialysis buffer were analyzed for elemental composition. The concentration values shown below were subtracted from values obtained for the dialysis buffer sample.

### Example 22

### Identification of the Chromophore Compound as Oxidized N-acetyldopamine (NADA)

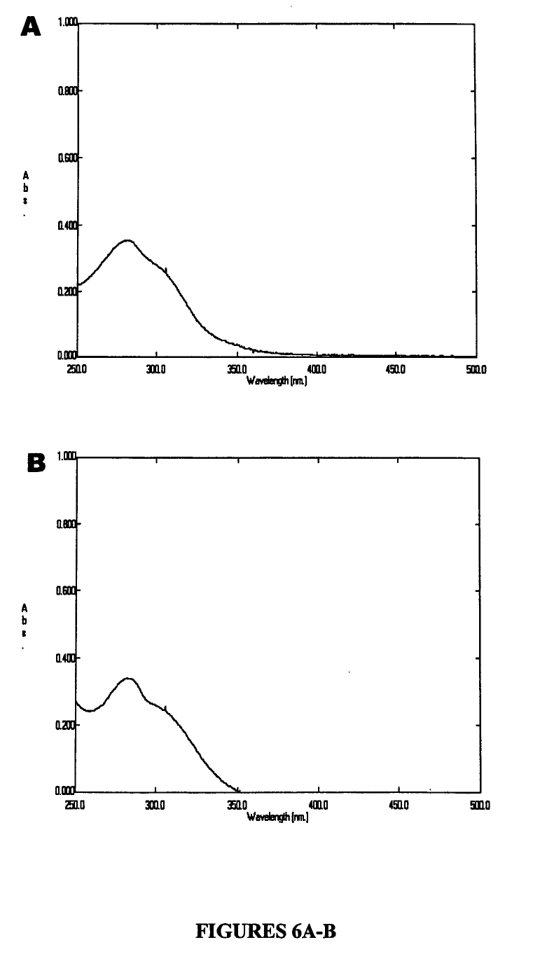

The mass of the chromophore does not correspond to the presence of either a folate compound or folate degradation product, because these molecules have greater masses than the observed mass of the chromophore. Dialysis of recombinant mouse MTHFS protein was not sufficient to remove the chromophore from the protein, indicating that the compound has a high affinity for the MTHFS protein. However, the chromophore was released from the purified recombinant MTHFS protein in the presence of 5-formylTHF. Application of the protein to a 5-formylTHF-sepharose column released the chomophore from the protein. The chromophore was collected in the flow-through fraction and UV spectrum recorded (FIG. 6A). The flow-through fraction did not contain protein, which was determined by spotting the sample on a nitrocellulose membrane and staining with Coomassie blue. The chromophore compound could also be removed from the protein by extraction with acetonitrile, which precipitated the protein.

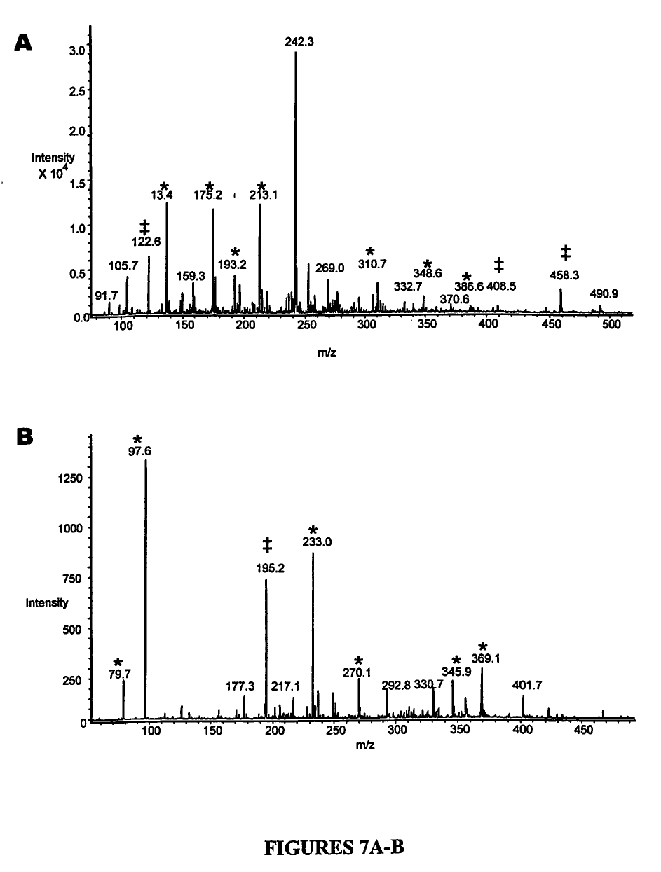

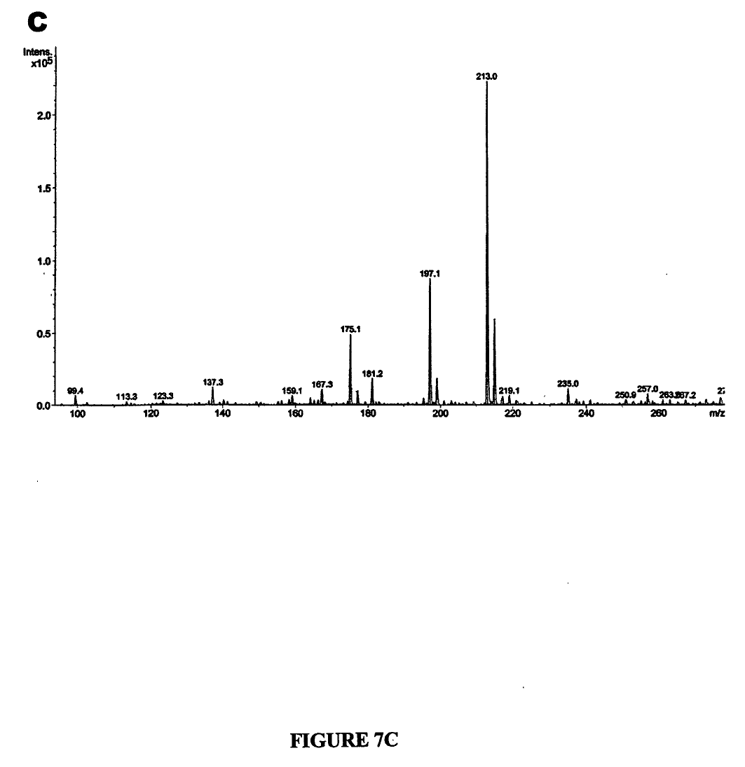

Removal of the chromophore by either isolation method allowed for further analysis using electrospray ionization mass spectroscopy (ESI-MS). The ESI-MS spectrum of the chromophore (FIG. 7) resembled that of known catecholamine standards. Peaks labeled with an asterisk represent peaks that were also observed in the mass spectra of an oxidized N-acetyldopamine (NADA) standard, and those labeled with crosses differ by one atomic unit from peaks present in the mass spectra of NADA. The molecular mass of NADA is 195 Da, and the positive mass spectrum of the chromophore in FIG. 7C contains a peak at m/z of 197 consistent with the presence of this catecholamine. The peak at m/z=213 in the positive mass spectra of the chromophore (in FIGS. 7A and 7C) is consistent with a NADA-H₂O compound or 6-hydroxy-NADA (molecular weight=211 Da), a catecholamine derivative that is formed at low pH (Garcia-Moreno et al., Arch. Biochem. Biophys. 288:427-434 (1991), which is hereby incorporated by reference in its entirety). The peak at m/z=363 is consistent with a NADA derivative that has dimerized, and this peak is also present in the positive mass spectrum of NADA standards. There are also peaks present in the chromophore spectra that are not present in the spectrum of NADA, which indicates that either there are different compounds in addition to NADA that are co-purified with the recombinant mouse MTHFS protein, or that the chromophore is a modified derivative of NADA. Alternatively, some of these peaks could be artifacts arising from the ESI-MS analysis, because compounds present in the sample can undergo gas phase chemistry during analysis, generating a greater number of chemical species (which would not represent true compounds in the original sample). Alternatively, additional compounds may have been generated during the removal of the chromophore from the protein. Evidence for this hypothesis is shown in FIG. 5; the positive mass spectrum of the chromophore removed using the 5-formylTHF-sepharose column (FIG. 7A) is slightly different from the spectrum of the chromophore removed from the protein by acetonitrile extraction (FIG. 7C).

The chromophore was analyzed using electrospray tandem mass spectroscopy (MS/MS) to determine its collision-induced dissociation pattern. The resulting peaks were compared to the MS/MS fragmentation pattern for a standard of NADA. Three peaks present in the chromophore sample (m/z=197, 213, 363) were selected for MS/MS fragmentation, and the resulting peaks for each fragmentation are shown in Table 5.

TABLE 5
Tandem mass spectrometry (ES-MS/MS) analysis
of the chromophore removed from recombinant
mouse MTHFS and a standard of NADA.

| MTHFS | NADA | MTHFS | NADA | MTHFS | NADA |
|---|---|---|---|---|---|
| (m/z = | (m/z = | (m/z = | (m/z = | (m/z = | (m/z = |
| 363) | 363) | 197) | 197) | 213) | 213) |
| 363.2 | 363.2 | 197 | 196.3 | 213.4 | 213.3 |
| 214.4 | 214.3 | 179.3 | 179.3 | 196.2 | 197.3 |
| 185.4 | 185.3 | 167.3 | / | 185.2 | 185.3 |
| 174.4 | 174.4 | 155.2 | 154.3 | / | 171.3 |
| 146.5 | 146.4 | 137.3 | 137.4 | 88.6 | / |
| 130.5 | 130.4 | 123.5 | / | | |

The predominant peaks (m/z = 197, 213, 363) present in the positive mass spectrum of the chromophore (removed using acetonitrile extraction) were selected for MS/MS analysis (labeled as “MTHFS”), and the resulting fragmentation peaks are shown. Peaks present in a sample of NADA (m/z = 197, 213, 363) were also fragmented using MS/MS analysis, and the resulting peaks are shown.

Interestingly, the MS/MS fragmentation of three peaks present in a sample of NADA (m/z=197, 213, 363) yielded nearly identical fragmentation patterns as the chromophore sample (Table 5). The results provide strong evidence that the MTHFS-bound chromophore is a NADA derivative. Furthermore, the UV spectrum of NADA oxidized with sodium periodate at pH 7.0 yielded an identical UV spectrum as that for the chromophore removed from the protein using the 5-formylTHF-sepharose column (FIG. 6).

### Example 23

### Reconstitution of MTHFS Proteins with NADA

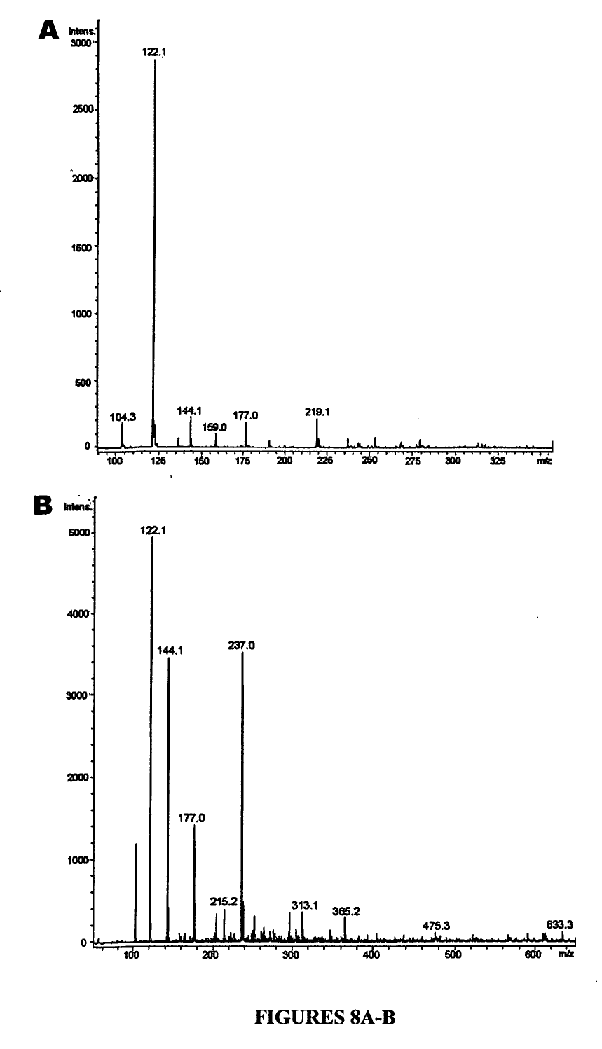

Bacteria are not known to synthesize catecholamines, indicating that the MTHFS-bound chromophore originated from the culture medium. To test this hypothesis, E. coli expressing the murine MTHFS cDNA were grown in M9 minimal media containing and/or lacking NADA, and the chromophore compound was removed from the protein and analyzed by ESI-MS. Recombinant mouse MTHFS protein grown in M9 minimal media without NADA does not contain the oxidized NADA derivative (FIG. 8A). The spectrum of the MTHFS-bound chromophore removed from the protein expressed in E. coli in M9 medium containing 10 mg NADA contains four peaks (m/z=215, 237, 313, 365) that are not present in the spectrum of the flowthrough fractions from protein expressed in M9 medium lacking NADA (FIG. 8). Two of these peaks (m/z=215 and 237) are similar in size to peaks in the positive mass spectra of the chromophore (FIGS. 7A and 7C), and the other two peaks are absent, indicating that NADA has likely undergone chemical transformations while in the culture medium or after transport into E. coli. The same experiment was repeated using M9 minimal media containing [¹³C]-tyrosine, which is a precursor of dopamine and NADA (Rescigno et al., Biochemical Pharmacology 56:1089-1096 (1998), which is hereby incorporated by reference in its entirety), and there was no difference between the mass spectrum of the control sample (FIG. 8A) and the spectrum of the flowthrough fraction after the labeled protein was loaded on a 5-formylTHF-sepharose column. Therefore, the enzymes needed to convert tyrosine into dopamine and NADA are not present in E. coli. However, these results do demonstrate that NADA present in the culture medium is taken up by E. coli and undergoes chemical and perhaps enzymatic transformation, and these NADA-like compounds retain the ability to co-purify with recombinant mouse MTHFS.

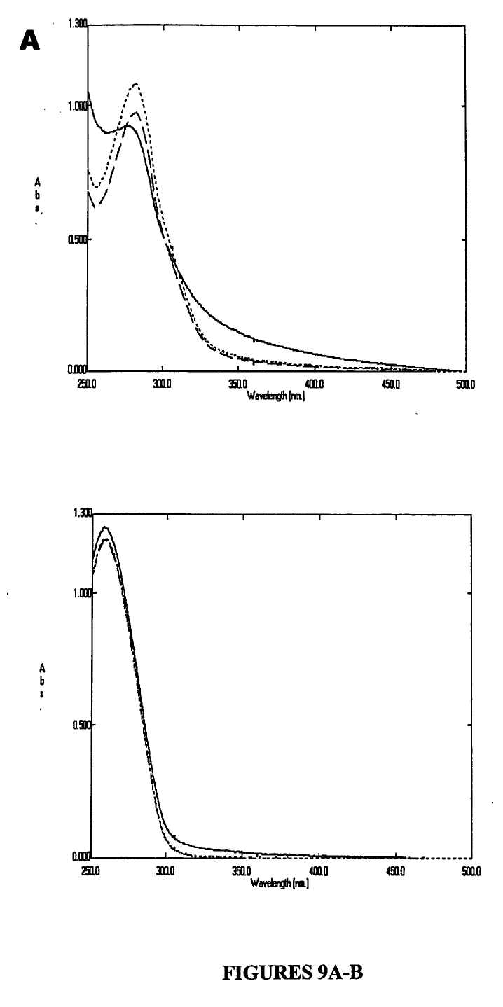

The UV spectrum of purified rabbit liver MTHFS differs from the spectrum of recombinant mouse MTHFS (FIG. 4). Therefore, it was investigated whether rabbit liver MTHFS could be reconstituted with NADA resulting in a change in the UV spectrum of the protein. Catecholamines rapidly oxidize, generating highly reactive quinone and semiquinone compounds that undergo nucleophilic addition reactions, yielding a variety of different chemical species (Rescigno et al., Biochemical Pharmacology 56:1089-1096 (1998), which is hereby incorporated by reference in its entirety). Therefore, reconstitution studies using oxidized NADA compounds are hindered by the short half-life of oxidized catecholamines in solution and the ability of these compounds to oxidize and chemically modify proteins (protein side chains will covalently bind catecholamine quinones) (Pattison et al., Toxicology 177:23-37 (2002); Graham, D. G. Mol. Pharmacol. 14:633-643 (1978), which are hereby incorporated by reference in their entirety). Purified rabbit liver MTHFS was incubated with NADA, oxidized NADA, and a phosphate buffer control for 15 min, then the protein samples were dialyzed overnight. Incubation of rabbit liver MTHFS with oxidized NADA resulted in a change in the UV spectrum of the protein relative to the control sample, and the absorbance increase occurred in the 300-450 nm range (FIG. 9A). The experiment was repeated using recombinant mouse MTHFS protein, and incubation with oxidized NADA resulted in the amplification of the shoulder peak absorbance in the UV spectrum of the protein. Incubation of yeast aldehyde dehydrogenase with NADA, oxidized NADA, and phosphate buffer did not change the UV spectra of the protein (FIG. 9B). The MTHFS-bound chromophore could be removed by applying the protein to a 5-formylTHF-sepharose column, indicating that the oxidized NADA was not covalently bound to the MTHFS protein and that oxidized NADA does not oxidize or modify the MTHFS protein. NADA did not inhibit the MTHFS-catalyzed conversion of 5-formylTHF to 5,10-methenylTHF; the ability of oxidized NADA to inhibit this reaction was not able to be determined because oxidized NADA interferes with the spectrophotometric assay used to quantify MTHFS activity (Girgis et al., Journal of Biological Chemistry 272:4729-4734 (1997), which is hereby incorporated by reference in its entirety). In summary, purified rabbit liver MTHFS can be reconstituted with oxidized NADA, and MTHFS proteins from different sources bind oxidized NADA with specificity. Furthermore, there is some evidence that oxidized NADA binds at a site other than the folate binding site of the protein as evidenced by its release from the protein upon binding 5-formylTHF and the inability of NADA to inhibit the MTHFS-catalyzed conversion of 5-formylTHF to 5,10-methenylTHF.

### Example 24

### MTHFS-Mediated Folate Catabolism In Vitro

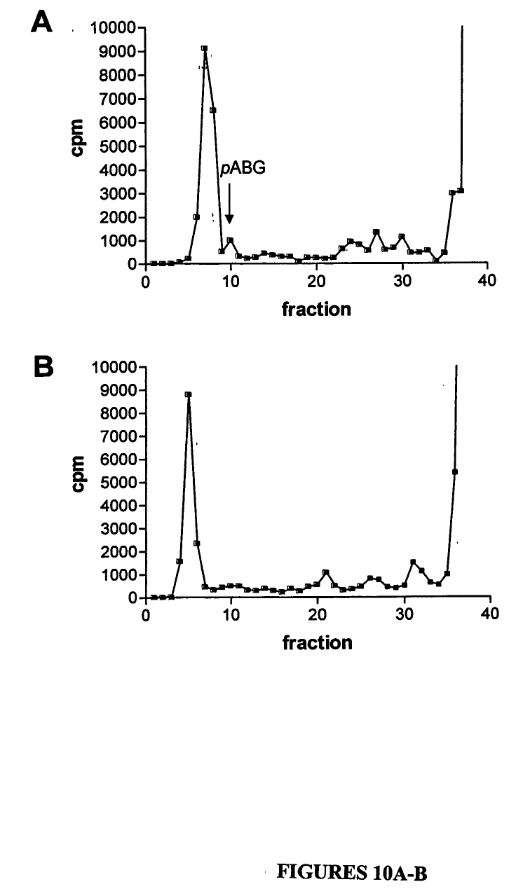

The ability of the MTHFS.oxNADA.Cu²⁺ complex to catalyze folate catabolism in vitro was investigated. The recombinant mouse MTHFS protein was pre-incubated with CuCl₂ and oxidized NADA for 15 min, then dialyzed for 4-5 h in Tris buffer to remove any unbound Cu²⁺ or oxidized NADA that could oxidize the reduced folate derivatives independent of MTHFS protein. Catabolism reactions were initiated by the addition of radiolabeled folates known to bind to the dialyzed MTHFS protein (either 5-formylTHF or 5-methylTHF), and the reaction mixture was incubated for 5 min. Following incubation, the reaction mixture was analyzed immediately for the presence of pABG by HPLC using a method described previously (Anguera et al., J. Biol. Chem. 278:29856-29862 (2003), which is hereby incorporated by reference in its entirety). Control reactions were performed that lacked MTHFS protein but contained equal volumes of the Tris buffer used to dialyze the reconstituted mouse MTHFS protein. (6S)-5-formylTHF was not catabolized in either the control samples or samples containing the reconstituted MTHFS protein. However, incubation of stoichiometric concentrations of MTHFS and (6S)-5-methylTHF, which has a Kᵢ of 18 μM for MTHFS (Bertrand et al., Biochim Biophys Acta 911:154-161 (1987), which is hereby incorporated by reference in its entirety), resulted in the generation of pABG, indicative of folate catabolism (FIG. 10). Approximately 1% of the (6S)-[³H]-5-methylTHF was converted to pABG by the reconstituted mouse MTHFS.oxNADA.Cu²⁺ protein in five independent experiments. The identity of pABG was verified by spiking the reaction samples with unlabeled pABG (elutes at t=10 min) and 5-methylTHF (elutes at t=37 min) prior to HPLC analysis, and this verified the peak at t=10 min as pABG (FIG. 10). This result is noteworthy, because the oxidation of 5-methylTHF, which can occur in the absence of reducing agents or by incubation with oxidized NADA alone, generates 5-methylDHF without any production of pABG (Lewis et al., Anal. Biochem. 93:91-97 (1979); Maruyama et al., Anal. Biochem. 84:277-295 (1978), which are hereby incorporated by reference in their entirety). Oxidation of 5-methylTHF to 5-methylDHF occurred in all the reaction samples because of the lack of a reducing agent, but pABG was generated in the MTHFS-containing reactions. The presence of Cu²⁺, MTHFS, and oxidized NADA was essential for the generation of pABG, and reactions containing MTHFS protein that was reconstituted with either Cu²⁺ or oxidized NADA alone did not generate pABG. These results provide evidence that MTHFS protein containing both Cu²⁺ and oxidized NADA can degrade folate in vitro albeit at low efficiency, and this supports the hypothesis that the MTHFS protein catalyzes folate catabolism.

### Example 25

### Effect of Catecholamines on Folate Turnover and Folate Accumulation

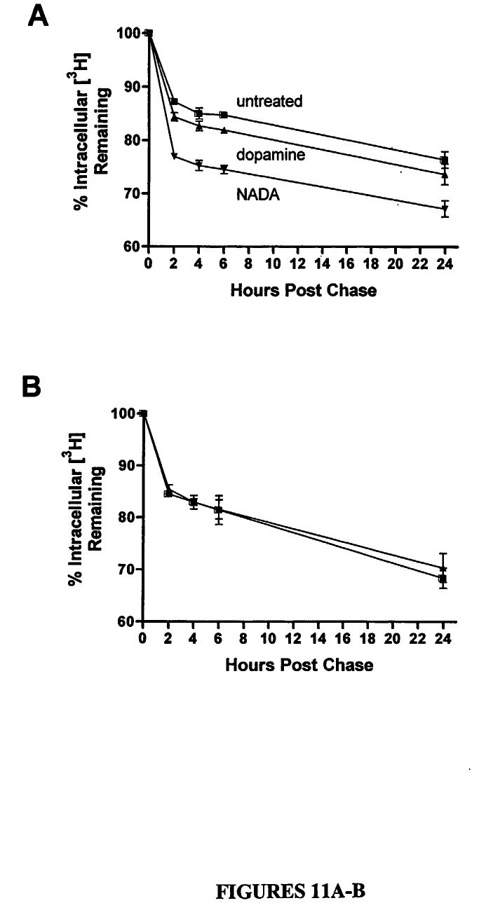

Catecholamine availability may be rate limiting for MTHFS-catalyzed folate catabolism in MCF-7 cells. The effects of exogenous catecholamines on folate turnover rates were determined using pulse-chase analyses in MCF-7 cells. Folate turnover in MCF-7 cells labeled with [³H]-5-formylTHF is biphasic, with an initial rapid phase of turnover followed by a slower phase (Anguera et al., J. Biol. Chem. 278:29856-29862 (2003), which is hereby incorporated by reference in its entirety). Addition of 20 μM NADA to the chase medium increased the magnitude of the rapid phase of folate turnover by 12% compared to untreated cells (FIG. 11A). The slow phase of folate turnover of MCF-7 cells treated with NADA did not change relative to untreated cells (FIG. 11A). Previous studies have indicated that the rapid phase of turnover represents the catabolism of newly imported short chain folate polyglutamates, and the slower phase represents degradation of the longer-chain folate polyglutamates (Anguera et al., J. Biol. Chem. 278:29856-29862 (2003), which his hereby incorporated by reference in its entirety). This result indicates that that NADA availability may be limiting for folate catabolism in vivo. Alternatively, this result could indicate that NADA, and other catecholamines such as dopamine and L-dopa, can undergo oxidation and accelerate folate catabolism in vivo independent of MTHFS. To distinguish between these two mechanisms, 20 μM dopamine and L-dopa were added to the chase medium. Dopamine is the precursor of NADA, and both dopamine and L-dopa can function as oxidants following oxidation of their diphenolic ring generating dopamine-quinone or dopa-quinone (Rescigno et al., Biochemical Pharmacology 56:1089-1096 (1998); Graham, D. G. Mol. Pharmacol. 14:633-643 (1978), which are hereby incorporated by reference in their entirety). Results from equilibrium binding studies using [³H]-dopamine demonstrated that neither dopamine nor oxidized dopamine bind to recombinant mouse MTHFS protein. The addition of dopamine to the culture medium increased the magnitude of the rapid phase of folate turnover by only 4% relative to untreated cells (FIG. 11A). The minor effect of dopamine on folate turnover may reflect N-acetyltransferase-mediated dopamine acetylation that generating NADA. The addition of L-dopa to the chase media did not affect folate turnover rates compared to untreated MCF-7 cells (FIG. 11B). These results demonstrate that not all catecholamines are capable of increasing folate turnover rates or that catecholamines differ in their ability to accumulate in MCF-7 cells. In summary, the pulse chase experiments indicate that NADA may be limiting for in vivo folate catabolism, and NADA may function specifically as the electron acceptor molecule for MTHFS-mediated folate catabolism.

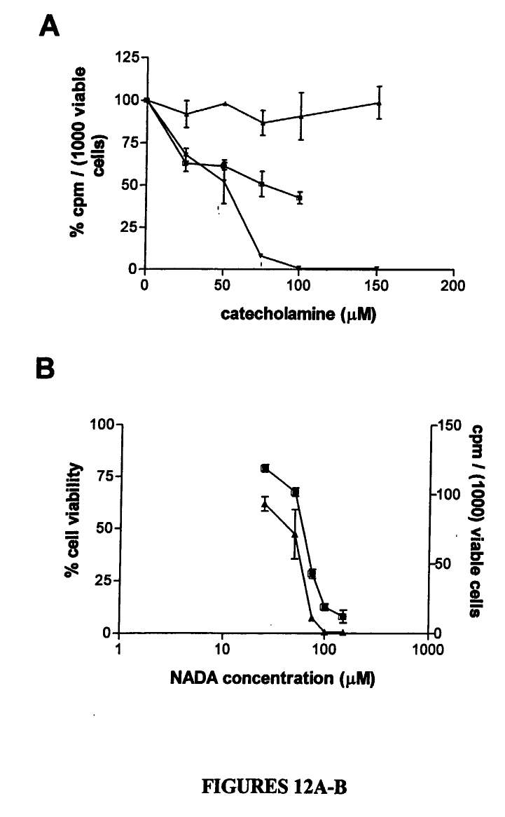

Because the addition of NADA to the chase medium increased folate turnover rates in MCF-7 cells, the effects of various catecholamines on [³H]folate accumulation was investigated. Cells were cultured for 12 h in defined αMEM medium containing 25 nM (6S)-[³H]-5-formylTHF and various concentrations of NADA, dopamine, and epinephrine, and cellular [³H]folate content was determined. Once transported into the cell, [³H]-5-formylTHF does not accumulate, because it equilibrates rapidly into the folate cofactor pool through the catalytic activity of MTHFS (Girgis et al., J Biol Chem 272:4729-4734 (1997), which is hereby incorporated by reference in its entirety). Treatment of MCF-7 cells with increasing concentrations of NADA decreased [³H]folate accumulation in a dose-dependent manner (FIG. 12A). These results support the data from pulse-chase experiments, suggesting that NADA is rate-limiting in MTHFS-mediated folate catabolism. Analysis of MCF-7 cell viability following incubation with various concentrations of NADA indicates that the dose-dependent decreases in cellular [³H]folate parallel NADA-induced cell death (FIG. 12B). Neither the addition of dopamine nor epinephrine to culture media impaired [³H]folate accumulation as observed for NADA (FIG. 12A). These results indicate that catecholamines that are capable of undergoing rapid oxidation (such as dopamine and epinephrine) do not degrade [³H]folate in cultured cells, and that the specific effect of NADA on cellular folate turnover and accumulation may result from its binding to MTHFS and generating an MTHFS.oxNADA.Cu²⁺ complex that catalyzes folate catabolism.

The experiments described in this study investigated the ability of MTHFS to catalyze the oxidative degradation of folate. The oxidative degradation of folate requires a two-electron acceptor that is capable of binding to MTHFS. UV and MS analysis of the recombinant protein indicated that a small molecule co-purifies with the protein, and extensive ESI-MS analysis determined that this compound is oxidized NADA or a derivative thereof. The ortho-diphenolic groups present in catecholamines such as NADA and dopamine undergo rapid oxidation generating an ortho-quinone species, which is short lived and very reactive (Rescigno et al., Biochemical Pharmacology 56:1089-1096 (1998), which is hereby incorporated by reference in its entirety). Dopamine and NADA quinones are known to function as two-electron acceptors for a variety of redox reactions in vitro, which regenerates the diphenolic ring (Garcia-Moreno et al., Arch. Biochem. Biophys. 288:427-434 (1991); Graham, D. G. Mol. Pharmacol. 14:633-643 (1978), which are hereby incorporated by reference in their entirety). The oxidized NADA derivative that co-purifies with recombinant mouse MTHFS, in the quinone form, may function as the oxidant molecule required for the two-electron oxidation in folate catabolism. The recombinant mouse MTHFS protein also co-purifies with non-stoichiometric amounts of Cu²⁺, and the sub-stoichiometric concentrations of copper may have resulted from the use of the metal affinity resin and extensive dialysis that was used to purify the recombinant protein. There are numerous examples of proteins containing quinone cofactors and metal ions that are known to catalyze oxidative reactions. For example, copper amine oxidases contain a cupric ion that is in close proximity to a quino-cofactor, tyrosine-derived 2,4,5-trihydroxyphenylalanyl quinone, and these enzymes catalyze an oxidative deamination of amines to aldehydes (Klinman, J. P. Biochimica et Biophysica Acta 1647:131-137 (2003), which is hereby incorporated by reference in its entirety). Both Cu²⁺ and oxidized NADA co-purified with recombinant mouse MTHFS protein, and both compounds were necessary to reconstitute MTHFS for 5-methylTHF catabolism to pABG in vitro.

This is the first study to demonstrate that the MTHFS protein, in the presence of oxidized NADA and Cu²⁺, can catabolize (6S)-5-methylTHF to pABG in vitro. The oxidation of reduced folate derivatives have been studied extensively, and the oxidation of 5-methylTHF is known only to generate 5-methylDHF as the final product (Lewis et al., Anal. Biochem. 93:91-97 (1979); Maruyama et al., Anal. Biochem. 84:277-295 (1978), which are hereby incorporated by reference in their entirety). Initial attempts to demonstrate the in vitro catabolic activity of MTHFS were difficult, because oxidized NADA can oxidize reduced folate derivatives in vitro, resulting in high background. Dialysis of the MTHFS protein reconstituted with oxidized NADA and Cu²⁺ eliminated most non-enzymatic or background folate oxidation. Two reduced folate derivatives that are known to bind to MTHFS were tested as substrates for folate catabolism, and only 5-methylTHF was catabolized to pABG in the presence of the MTHFS protein that was reconstituted with both Cu²⁺ and oxidized NADA. SH-SY5Y neuroblastoma cells with increased MTHFS activity have decreased cellular 5-methylTHF levels compared to nontransfected cells (Girgis et al., Journal of Biological Chemistry 272:4729-4734 (1997), which is hereby incorporated by reference in its entirety). SH-SY5Y neuroblastoma cells are known contain both catecholamines and tyrosinase, which is capable of oxidizing catecholamines including NADA (Higashi et al., Journal of Neurochemistry 75:1771-1774 (2000); Song, X. E. International Journal of Toxicology 17:677-701 (1998), which are hereby incorporated by reference in their entirety). Therefore, MTHFS.oxNADA.Cu²⁺-mediated catabolism may account for the decrease in cellular 5-methylTHF in SH-SY5Y cells with increased MTHFS activity (Anguera et al., J. Biol. Chem. 278:29856-29862 (2003); Girgis et al., Journal of Biological Chemistry 272:4729-4734 (1997), which are hereby incorporated by reference in their entirety).

The data presented in this study suggest that NADA availability is rate-limiting for MTHFS.oxNADA.Cu²⁺-mediated folate catabolism in MCF-7 cells because increased concentrations of NADA accelerated folate turnover and decreased folate accumulation. NADA is detected in the urine of mammals under normal conditions and when treated with monoamine oxidase inhibitors (Hanson et al., Clin. Chim. Acta 11:384-385 (1965), which is hereby incorporated by reference in its entirety). Children with neuroblastomas and nephroblastomas display 13-fold and 4-fold, respectively, higher levels of urinary NADA compared to unaffected individuals (Hanson et al., Clin. Chim. Acta 11:384-385 (1965); Jouve et al., Journal of Chromatography 574:9-15 (1992), which are hereby incorporated by reference in their entirety). The work presented here suggests that individuals with neuroblastomas and nephroblastomas may have increased folate catabolism rates and display symptoms of folate deficiency, and future studies investigating levels of urinary pABG in these individuals are warranted.

In vitro catabolism of (6S)-5-methylTHF by the MTHFS.oxNADA.Cu²⁺ complex resulted in approximately 1% conversion of 5-methylTHF to pABG. The efficiency of the reconstituted MTHFS catabolism reaction may be low because: (1) the MTHFS catalyzed degradation of folate displays specificity for an oxidized NADA derivative that was not enriched in the preparation of oxidized NADA, and/or (2) non-catalytic forms of oxidized NADA (including oligomeric forms) bind competitively to MTHFS with the catalytically-competent oxidized NADA, and/or (3) 5-methylTHF may be a poor substrate for MTHFS-mediated catabolism. Catecholamine quinone compounds are unstable and are susceptible to undergo nucleophilic attack by molecules such as hydroxide ions, primary amines, cysteinyl residues on proteins (and 2-mercaptoethanol), and other cellular nucleophiles (Rescigno et al., Biochemical Pharmacology 56:1089-1096 (1998), which is hereby incorporated by reference in its entirety). O-quinone cyclization can also occur via nucleophilic attack by the amine group of the side chain of dopamine on the diphenolic ring (cyclization prevails at pH>6), and this results in an unstable o-quinoid indolic compound that can undergo further covalent modifications by nucleophiles (Rescigno et al., Biochemical Pharmacology 56:1089-1096 (1998), which is hereby incorporated by reference in its entirety). The in vitro catabolism assay utilized NADA that was chemically oxidized with sodium periodate, and either chemical or enzymatic oxidation of NADA is known to generate a NADA-quinone that quickly transforms into o-quinoid indolic compounds that dimerize eventually forming melanins (Rescigno et al., Biochemical Pharmacology 56:1089-1096 (1998); Sugumaran et al., Archives of Insect Biochemistry and Physiology 8:229-241 (1988); Sugumaran, M. Arch. Biochem. Biophys. 378:404-410 (2000); Li et al., Journal of Electroanalytical Chemistry 375:219-231 (1994), which are hereby incorporated by reference in their entirety).

Therefore, it is possible that only a small amount of the appropriate oxidized NADA compound was able to bind to the mouse MTHFS before undergoing chemical conversion to other NADA derivatives that are unable to bind to the protein and/or function as the two-electron acceptor for MTHFS-mediated folate catabolism. Another reduced folate derivative, DHF, has been shown to bind tightly to MTHFS, and may also be a substrate for MTHFS.oxNADA.Cu²⁺-mediated folate catabolism. However, DHF is very unstable and degrades rapidly to pABG in the absence of reducing agents (Maruyama et al., Anal. Biochem. 84:277-295 (1978), which is hereby incorporated by reference in its entirety), and it was not possible to differentiate between non-enzymatic and enzymatic catabolism of this folate derivative.

Examination of the absorption intensities of the MALDI-TOF mass spectrum of recombinant mouse MTHFS protein indicated that approximately 41% of the total pool of MTHFS protein contains the presence of oxidized NADA (FIG. 5). Interestingly, the UV spectrum for recombinant Arabidopsis MTHFS (expressed in E. coli with a hexa-histidine tag) contains the chromophore shoulder peak at 320 nm similar to the recombinant mouse MTHFS protein, indicating that other recombinant MTHFS proteins also co-purify with a bound catecholamine. The UV spectrum of the purified rabbit liver MTHFS protein did not contain NADA, but this purification requires the use of a 5-formylTHF-sepharose affinity column, which removes NADA from the MTHFS protein. The reversibility of NADA binding in the presence of 5-formylTHF demonstrates that NADA was not bound covalently to the MTHFS protein. The data demonstrate that oxidized NADA binds tightly to MTHFS in the absence of 5-formylTHF, but that 5-formylTHF binding decreases the affinity of NADA for the protein. These results suggest that NADA has an independent binding site and that, upon binding the folate substrate, MTHFS undergoes a conformational change that alters its affinity for oxidized NADA. Determination of the three-dimensional structure of MTHFS will establish the architecture of the MTHFS active site, including the NADA binding site, which will support a mechanism for MTHFS.oxNADA.Cu₂₊-mediated catabolism of folate.

### Example 26

### MTHFS Binds 10-formyltetrahydrofolate and Enhances de novo Purine Biosynthesis

Inhibition of MTHFS by 10-Formyltetrahydrofolate.

Several 10-formyl-substituted folates were screened for their ability to inhibit wild-type MTHFS. The results are summarized in Table 6 below.

TABLE 6
Reaction Specificity of MTHFS¹

| | | |
|---|---|---|
| Folate/antifolate | Kᵢ(μM) | Kₘ(μM) |

Substrate

| | |
|---|---|
| (6RS)5-formylTHPteroate | 33 ± 10 |
| (6S)5-formylTHF | 10 ± 3 |
| (6RS)5-formylTHFGlu₃ | 0.4 ± 0.1 |

Inhibitor

| | | |
|---|---|---|
| 5-formyltetrahydropterin | N.D. | |
| folic acid | 58 ± 5 | — |
| folic acidGlu₃ | 17 ± 3 | — |
| folic acidGlu₅ | 1.0 ± 0.3 | |
| 10-formylfolic acid | 13 ± 6 | — |
| (6RS)5-formyl,10-methylTHF | 10 ± 1 | — |
| (6RS)5-formyl,10-methylTHFGlu₃ | 20 ± 2 | — |
| (6RS)10-formylTHPteroate | 5 ± 2 | — |
| (6R)10-formylTHF | 0.15 ± 0.9 | — |
| (6RS)10-formylTHFGlu₃ | 0.03 ± 0.01 | — |
| (6RS)5-formylTHHF | 0.7 ± 0.3 | — |
| (6RS)5-formyl,10-methylTHHF | 6.0 ± 3 | — |
| (6RS)10-formylTHHF | 0.19 ± 0.02 | — |

¹All reactions performed at 37° C., pH 6.0

N¹⁰ formylation of folic acid increased its affinity for MTHFS by nearly 5-fold, and 10-formylTHF binds with 56-fold increased affinity (Kᵢ) compared to the substrate 5-formylTHF (Kₘ), 10-formylTHF triglutamates bind with 13-fold increased affinity (Kᵢ) compared to the substrate 5-formylTHF triglutamate (Kₘ), and the Kᵢ for 10-formylTHHF is 3-fold lower than the Kᵢ for 5-formyltetrahydrohomofolate (5-formylTHHF), a known MTHFS inhibitor. In summary, (6R)10-formylTHF is a naturally-occurring and more effective inhibitor than the synthetic antifolate MTHFS inhibitor, 5-formylTHHF.

Folate binding proteins can serve as “sinks” that sequester certain folates and thereby inhibit certain pathways, or can interact with other enzymes to selectively “channel” co-factors and accelerate individual biosynthetic pathways. 10-formylTHF is required for purine biosynthesis catalyzed by the enzymes GARFT and AICARFT. To determine the metabolic effects of 10-formylTHF sequestration on purine biosynthesis, a “formate suppression” assay was developed in which mammalian cells expressing MTHFS cDNA were cultured in the presence of [³H]-hypoxanthine and [¹⁴C]-formate. [³H]-Hypoxanthine is converted to purines via the folate-independent salvage pathway, whereas [¹⁴C]-formate is incorporated into purines via the de novo pathway after condensing with THF. The ability of the de novo purine biosynthetic pathway to suppress contributions from the purine salvage pathway to DNA synthesis was investigated in human SHSY-5Y and SHSY-5YMTHFS neuroblastoma cells. SHSY-5YMTHFS cells display 100-fold increased MTHFS activity and protein level. The ratio of ¹⁴C to ³H (dpm) in DNA and purine nucleotides serves a measure of de novo purine synthesis efficiency. The ¹⁴C/³H ratio is two-fold higher in SHSY-5YMTHFS genomic DNA compared to the parent cell line. Because ¹⁴C could also be incorporated into deoxythymidine and methylcytsosine (FIG. 2) via equilibration into the one-carbon pool, the DNA was digested to nucleosides, which were fractionated by HPLC. The deoxyguanosine and deoxyadenosine ¹⁴C/³H ratio was increased by 43% and 69% in SHSY-5YMTHFS cells compared to the parent cell line. In contrast, comparison of ¹⁴C counts derived from purified deoxythymidine normalized to ³H counts from deoxyadenosine in the two cell lines indicated that increased MTHFS expression impairs de novo dTMP synthesis. The enhancement of purine biosynthesis by increased MTHFS expression suggests that MTHFS shuttles bound 10-formyTHF to de novo purine synthesis. The data in Table 6 indicate that high-affinity MTHFS inhibitors should be N10-formyl-substituted and be capable of conversion to polyglutamate derivatives in the cell.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method of screening test substances for chemotherapeutic activity or efficacy in treating cardiovascular disease, said method comprising:
providing one or more cells transformed with a nucleic acid molecule encoding methenyltetrahydrofolate synthetase;
contacting the cells with one or more test substances; and
identifying the test substances which modulate methenyltetrahydrofolate synthetase expression by the cells as candidate substances for chemotherapeutic activity or efficacy in treating cardiovascular disease.

2. The method according to claim 1, wherein the test substance is screened for chemotherapeutic activity.

3. The method according to claim 1, wherein the test substance is screened for efficacy in treating cardiovascular disease.

4. The method according to claim 1, wherein said identifying determines when methenyltetrahydrofolate synthase expression is reduced.

5. The method according to claim 1, wherein said identifying determines when methenyltetrahydrofolate synthase expression is increased.

6. A method of measuring folate status in a sample, said method comprising:
providing a sample;
measuring methenyltetrahydrofolate synthetase expression or activity and/or catecholamine activity in the sample; and
correlating the measured level of methenyltetrahydrofolate synthetase expression or activity and/or catecholamine activity in the sample to folate status in the sample.

7. The method according to claim 6, wherein the sample is from a subject.

8. The method according to claim 7, wherein the sample is a tissue sample from the subject.

9. The method according to claim 7, wherein the sample is a bodily fluid sample from the subject.

10. The method according to claim 9, wherein the bodily fluid sample is selected from the group consisting of blood, serum, and urine.

11. The method according to claim 6 further comprising:
determining cancer state of the subject based on the folate status.

12. The method according to claim 6 farther comprising:
determining progression of cancer in the subject based on the folate status.

13. The method according to claim 12, wherein said determining is carried out by comparing early folate status measurements to subsequent folate status measurements in the subject.

14. The method according to claim 6 farther comprising:
dosing a chemotherapeutic agent based on the folate status.

15. The method according to claim 6 farther comprising:
determining cardiovascular disease state of the subject based on folate status.

16. The method according to claim 15, wherein said determining is carried out by comparing early folate status measurements to subsequent folate status measurements in the subject.

17. The method according to claim 6 further comprising:
dosing an agent for treatment of a cardiovascular disease based on folate status.

18. The method according to claim 6, wherein methenyltetrahydrofolate synthetase expression is measured in the sample and correlated to folate status in the sample.

19. The method according to claim 6, wherein methenyltetrahydrofolate synthetase activity is measured in the sample and correlated to folate status in the sample.

20. The method according to claim 6, wherein catecholamine activity is measured in the sample and correlated to folate status in the sample.

21. A method of treating or preventing cancer in a subject, said method comprising:
administering to the subject a substance which modulates methenyltetrahydrofolate synthetase expression or activity and/or catecholamine activity under conditions effective to treat or prevent cancer in the subject.

22. The method according to claim 21, wherein cancer is treated.

23. The method according to claim 21, wherein cancer is prevented.

24. The method according to claim 21, wherein methenyltetrahydrofolate synthetase expression is modulated by said administering.

25. The method according to claim 21, wherein methenyltetrahydrofolate synthetase activity is modulated by said administering.

26. The method according to claim 21, wherein catecholamine activity is modulated by said administering.

27. The method according to claim 21, wherein the cancer is selected from the group consisting of colon cancer, lymphoma, brain tumors, prostate cancer, multiple myeloma, breast cancer, and Wilms tumors.

28. The method according to claim 21, wherein said administering inhibits methenyltetrahydrofolate synthetase expression or activity or catecholamine activity.

29. The method according to claim 21, wherein said administering activates methenyltetrahydrofolate synthetase expression or activity or catecholamine activity

30. A method of treating or preventing cardiovascular disease in a subject, said method comprising:
administering to the subject a substance which inhibits methenyltetrahydrofolate synthetase expression or activity and/or catecholamine activity under conditions effective to treat or prevent cardiovascular disease in the subject.

31. The method according to claim 30, wherein cardiovascular disease is treated.

32. The method according to claim 30, wherein cardiovascular disease is prevented.

33. The method according to claim 30, wherein methenyltetrahydrofolate synthetase expression is modulated by said administering.

34. The method according to claim 30, wherein methenyltetrahydrofolate synthetase activity is modulated by said administering.

35. The method according to claim 30, wherein catecholamine activity is modulated by said administering.
